(19) United States
(12) Patent Application Publication
(10) Pub. No.: US 2020/0261182 A1
(43) Pub. Date: Aug. 20, 2020

(54) APPARATUS AND METHOD FOR TRACING PRIMARY AND PROCESS DEVICES, AND CLOSED STERILE TRANSFER FORMULATION AND FILLING IN CONNECTION WITH THE TRACED DEVICES

(71) Applicant: Dr. Py Institute LLC, New Milford, CT (US)

(72) Inventor: Daniel Py, Larchmont, NY (US)

(21) Appl. No.: 16/630,940

(22) PCT Filed: Jul. 14, 2018
(86) PCT No.: PCT/US2018/042196

§ 371 Date: Jan. 14, 2020

Related U.S. Application Data
(60) Provisional application No. 62/534,152, filed on Jul. 18, 2017.
(60) Provisional application No. 62/532,972, filed on Jul. 14, 2017.

(51) Int. Cl. A61B 90/98; A61M 5/14

(52) U.S. Cl. CPC A61B 90/98; A61M 39/10; A61M 5/14

(57) ABSTRACT

An apparatus and method for electronically tracing primary devices and process devices, and closed transfer formulation and/or filling the traced primary devices. Each of the primary devices and process devices includes an electronic identifier, such as an RFID tag or barcode. Scanners read the electronic identifiers, and transmit the read identification information to a controller. The controller compares the read identification information to required identification information for a respective product specification, and transmits a signal to further proceed with a formulation or filling process, or not, based on the comparison.

### CROSS-REFERENCE TO RELATED APPLICATIONS

This application claims benefit under 35 U.S.C. § 119(e) to similarly-titled co-pending U.S. provisional application No. 62/534,152 filed Jul. 18, 2017, and similarly-titled co-pending U.S. provisional application No. 62/532,972 filed Jul. 14, 2017, all of which are incorporated by reference in their entirety.

### FIELD OF THE INVENTION

The present invention relates to apparatus and methods for tracing primary and process containers and devices, and more particularly, to electronically tracing primary and process containers and devices during formulation and filling, such as by using RFID or barcodes, and/or to apparatus and methods that formulate and/or fill by closed sterile transfer where sterile substances are sealed with respect to, and transferred without exposure to the ambient atmosphere.

### BACKGROUND INFORMATION

Substances and products are often stored in, transported in, and dispensed from containers. Typically, a container has a body defining a chamber for storing the substance or product, and an opening through which the chamber is filled with the substance. After filling, the fill opening is often closed in some manner, such as by a cap or a closure, in order to keep the substance within the container. The typical filling process, then, involves filling the product into the open container through the fill opening, and then closing the fill opening.

For many products, it is important or desirable to limit the contaminants in the product stored in the container. Undesirable contaminants can include, for example, microbes, which can cause infections and reactions in living organisms. Excessive microbe growth can also change the characteristics of the product. Contaminants can also include non-living particulates and other substances. Though such non-living contaminants may not cause infections, they can cause adverse reactions in living organisms. Non-living contaminants may also adversely affect the characteristics of the substance, by the mere presence of the contaminants themselves, or in other ways, e.g., chemical reaction with the substance.

Contaminants are a particular concern for certain products used with or ingested by humans, animals, plants, and other living organisms. Examples of such products include foods, drinks, cosmetics, vaccines, medicines, pharmaceuticals, and sanitary and cleaning products. However, contaminants are not a concern merely with respect to products for living beings. It is a concern with respect to any product or industry in which contaminants can adversely affect the product or use of the product.

The traditional open filling process described above creates a critical opportunity for contamination to occur. Prior to filling, the internal chamber of the container is open to the environment, and contaminants can enter through the fill opening and contaminate the internal surfaces of the chamber. When the product is filled into the container and contacts the contaminated surfaces, contaminates can be transferred into the substance. In addition, during the filling process and until the container is closed, the substance itself is exposed to the environment, and can accumulate contaminates from the ambient atmosphere. Thus, even if the container and/or the substance are initially sterile and/or free of contaminants, the resulting filled product in the container may not remain in that condition.

Several approaches have been used to address the above-discussed concerns. One such approach is to include preservatives in the substance. Preservatives can be very effective in preventing microbial growth, reducing or eliminating living microbial presence in the substance, and preventing, reducing or slowing down degradation or spoilage of the substance. Preservatives have several disadvantages, though. Preservatives can react with the substance, reducing its effectiveness or efficacy. In the case of foods or drinks, preservatives can affect the taste. Some users have undesirable adverse reactions to certain preservatives. There is also growing concern that preservatives can have long-term adverse effects on the body, such as causing or promoting cancer, or are damaging to the environment, even if those preservatives are approved by regulatory authorities. In addition, though preservatives can reduce the risk of adverse contamination in the substance, such as by killing microbes, the contaminant materials are still present in the substance.

Another approach is to fill and close the container in an aseptic isolator. The isolator creates a barrier between the filling operation and the surrounding environment or ambient atmosphere. The conditions within the isolator are strictly controlled to ensure a filling environment that is sufficiently aseptic or sterile to prevent microbial contamination of the substance while filling the container, and also an environment with a specified maximum or classified level of other contaminants or particulates. Isolators thus help limit microbial and other contamination in the substance that is filled into the container. Isolators are commonly used in industries that require such characteristics of the substances produced. They are common in the pharmaceutical and medical industries, but also are used for certain non-medical foods and drinks, as well as products of other industries.

Isolator-type systems present certain drawbacks, however. The filling process is time consuming, and the processes and equipment are expensive. The personnel operating the isolators require particular training and equipment, such as isolation suits or protective clothing.

Further, the relatively complex nature of the filling processes and equipment can lead to more defectively filled containers than otherwise desired. For example, typically there are at least as many sources of failure as there are components. In many cases, numerous components must not only be sterilized and cleaned prior to the filling process, but then must be maintained sterile and clean. Any breach in or leakage through the isolation barrier can result in microbial and non-microbial contamination of the components and the filled substance.

Such isolators must also maintain the air within the barrier enclosure sterile and limited to the required level of particles. The isolators use expensive and complex air handling systems to filter and otherwise clean the air. A malfunction of the air systems may thus introduce contamination.

Yet another drawback is that such air handling systems can allow for the introduction of contaminants into the isolator. Air handling systems of this type typically employ high efficiency particulate air or “HEPA” filters to remove microorganisms. HEPA filters can remove particles, including microorganisms, with a diameter larger than 0.3 μm. However, such filters nevertheless can allow for contaminants, including germs and other microorganisms, to enter the isolator and, in turn, contaminate the components and/or filled substances. Even if the HEPA filters are working properly, such systems nevertheless can allow for the flow of contaminants into the isolators. For example, some such filters allow for about five colonies of germs per hour per square meter to pass into the isolator. Over time, such germs collect on the components and/or the open vials or other containers within the isolator and contaminate the surfaces with which they come into contact. Typically, the filling machines located within such isolators run at high speeds, in part, to minimize the exposure of the components and containers to such contaminants. However, high running speeds can lead to more frequent machine breakdowns and associated downtime than desired, which can lead to production delay and expense. In addition, during the downtime, the components and open containers may sit under the HEPA filters and be subjected to further contamination. Subjecting the open containers and components to, for example, five colonies of germs per hour per square meter can lead to unacceptably high levels of contamination.

Pharmaceutical and medical industries have frequently used glass containers, such as vials, for holding the pharmaceutical and medicinal products. Prior to filling in an isolator, such containers must be washed and depyrogenized, i.e., subject to depyrogenation. Depyrogenation requires passage of the glass containers through a depyrogenation tunnel where the containers are washed and then dried with air pumped through HEPA filters. After drying, the open containers are transported through the tunnels and/or along distribution tables that serve as buffers between the depyrogenation tunnels and the isolators. The open containers are subjected to an over-pressure of HEPA filtered air throughout such transportation. The time lag between depyrogenation and filling can vary from facility to facility, batch to batch, product to product, and/or vial to vial. In some cases, the open containers can remain on the distribution tables for extended periods of time, for example, on the order of several hours, such as about three hours. One drawback of such systems is that the HEPA filters can allow contaminants to pass through; for example, about five colonies of germs per hour per square meter. As a result, the critical surfaces of the containers, i.e., the surfaces that can come into contact with the pharmaceutical or medicinal product to be filled therein, can become contaminated and, in turn, contaminate the filled product. The longer the open containers are subjected to an over-pressure of HEPA filtered air, the greater is the likelihood and extent of contamination. In view of the foregoing, such purportedly sterile systems are not, in fact, sterile, but rather inherently subject the critical surfaces to contamination. Yet another drawback is the over-pressure causes the HEPA filtered air to flow through the openings in the containers and into contact with the critical surfaces, thus facilitating the deposit of contaminants onto the critical surfaces and the contamination of the products filled therein. The larger the openings in the containers, the greater the likelihood of critical surface contamination. As a result, such systems may require the filled products to be terminally sterilized in order to ensure that the filled-finished products are sterile. However, terminal sterilization can damage the pharmaceutical, medical or other product subjected to such sterilization processes, such as heat or radiation sterilization, and therefore is not desirable.

Often, a malfunction or barrier breach of the isolator systems will require the isolator to be shut down and repaired. The components must be adequately cleaned and sterilized prior to re-starting production. The safety and quality of any products manufactured and filled during the period of malfunction is also called into question, requiring disposal and possibly recall of the product. This can impose significant expense on the manufacturer. Moreover, if the malfunction is not detected or the time frame of the malfunction is not properly determined, contaminated product could remain on the market and used by customers, who could be injured or otherwise suffer losses from the contaminated product. For example, catheter related bloodstream infection from contaminated medical products kills can be particularly dangerous and can lead to death.

The risks are not limited to the filling process, but extend to manufacture of the product prior to filling. Many formulations are a combination of different ingredients that are blended or mixed together. The individual ingredients or the final product may also undergo additional processing, such as filtering, temperature treatments, etc. Typically, the ingredients and products must be transferred from one place to another during the process, often multiple places, such that the materials are transferred from and to a series of containers or vessels. For example, a product made by the blending of multiple ingredients would undergo at least the following:

(a) raw ingredients are transferred from their containers to a blending vessel;

(b) the ingredients are blended in the blending vessel to formulate the product;

(c) the formulated product is transferred to a filling machine; and

(d) the product is transferred from the filling machine into the final product container.

Each step of the process presents a risk of contamination, either from direct exposure of the ingredient or formulated product to the ambient atmosphere or environment, or by infiltration of contaminants into the vessels and transfer systems through which the ingredients and formulated product passes. Thus, under traditional formulation and filling methods, not only must the environment of the filling process itself be controlled, e.g., by using sterile isolators, but also the environment at every step of the process from raw ingredient storage to filling. Providing such a sterile or classified environment is complex, time-consuming and expensive.

Certain industries, such as the food and pharmaceutical industries, for example, are subject to regulatory control by governmental or industry authorities. In such cases, manufacturers must comply with applicable regulatory standards and controls. Often, the regulatory authority must inspect, certify or otherwise approve the production system for the product before production can commence, or in other circumstances, continue. Such regulatory audits and the preparations therefor can be significantly expensive and time-consuming events. All relevant controls must be compliant with the applicable regulatory guidelines, which can vary with the technology used to compound or otherwise manufacture the product. Typically, such guidelines are suggestions by the regulator and can be subject to varying interpretation depending on the regulatory auditor. As a result, it can be difficult to predict the outcomes of such regulatory audits. There is not believed to exist an auditor's checklist or like information that could be used to improve the predictability of the outcomes of such audits.

In the pharmaceutical industry, for example, the timeline to design, build and achieve regulatory approval of a drug production line is usually measured in months if not years. This extended timeline can have consequences. For example, if a product is needed quickly to address an urgent need, the product, or enough product, may not be delivered in time. One example of this would be if a natural disaster or other event disrupted the food or water supply to an area or population. Unless enough product can be made quickly enough, people may suffer or die. In such instances, the critical timeframe may be weeks, not months or years.

Another example, in a health context, would be an epidemic or pandemic outbreak, or a drug shortage. Needed vaccines or drugs may not be able to be produced quickly enough or in enough doses to prevent the spread of the disease and/or treat victims. The Spanish Flu pandemic in 1918 is believed to have infected 500 million people, and killed 50-100 million people, a 10-20% fatality rate. Yet it took months to spread around the world. Today, though, in view of current mobility of people and products, a similar pandemic would spread around the world in weeks, according to current propagation models. Distance from the outbreak would not necessarily provide protection. Propagation models predict, for example, that an outbreak in New York City would spread to Shanghai faster than to Trenton, N.J., based on current travel patterns and rate of population transfer.

Accordingly, experts predict that an outbreak today similar to the 1918 Spanish Flu outbreak would be significantly more disastrous. Billions could become infected. Hundreds of millions or more could die. Hospitals and medical facilities would be over-extended and over-crowded. Infection and death of medical personnel would create an acute shortage of medical care. The GDP of impacted countries could drop significantly, resulting in global economic crisis.

Unfortunately, the traditional system for manufacturing vaccines using aseptic isolators would present difficulties in responding to such a crisis. The time it takes design, build and obtain approval for such manufacturing systems (e.g., by the FDA) would delay introduction and production of needed medicines. Experts, both governmental and non-governmental, have concluded based on current data and models that a fully adequate or “just in time” response to pandemics is impossible using traditional technology. The U.S. Department of Homeland Security, for example, estimates that producing 50 million doses of a vaccine or drug using traditional manufacturing and processes would require one to two months. Such would be highly inadequate in the face of a pandemic that could infect hundreds of millions or more in that time frame. Many experts have concluded that new technologies for producing, filling and dispensing drugs and vaccines are absolutely necessary to adequately combat pandemics and drug shortages.

The need for improvement in safety and speed is not limited to pharmaceuticals, however. The need for improved safety and speed has been recognized across many diverse industries and products. To date, though, that need has not been fulfilled.

It is an object of the present invention to overcome one or more of the above described drawbacks and/or disadvantages of the prior art.

### SUMMARY OF THE INVENTION

In one aspect, a method comprises: (i) reading electronic identifiers on one or more primary devices or process devices; (ii) transmitting the read identification information to a controller, comparing the read identification information to required identification information for a respective specification, and transmitting a signal to further proceed or not based on the comparison; and (iii) if a signal to further proceed is transmitted, transferring by closed sterile transfer one or more substances from the primary device(s) to the process device(s), and/or from the process device(s) to the primary device(s).

In some embodiments, the identification information includes first information identifying the respective device and distinguishing the device from other devices. In some such embodiments, the identification information further includes second information on the condition or processing status of the respective device. In some such embodiments, the second information includes whether the respective device is sterile or was subjected to sterilization in a sealed, empty state.

In some embodiments, the primary devices include formulation component containers and formulation containers. In such embodiments, step (iii) includes transferring by closed sterile transfer a plurality of formulation components from respective component containers to a formulation container and combining the formulation components into a formulation in the formulation container. In some embodiments, the process devices include sterile connector assemblies. In some such embodiments, each sterile connector assembly includes a first connector and a second connector. The first and second connectors are connectable to each other and configured to transfer substance through the sterile connector assembly by closed sterile transfer. In some embodiments, each sterile connector assembly includes an electronic identifier, and is receivable within a respective connector support. The connector support includes a reader configured to read the electronic identifier of the sterile connector assembly. The method further comprises (i) transmitting a signal to the controller indicative of identification information of the respective sterile connector, (ii) comparing the identification information to required identification information for the respective support, and (iii) further proceeding or not based on the comparison. Some embodiments further comprise (i) measuring at the connector support a flow rate of a formulation or one or more formulation components flowing through the respective sterile connector, (ii) transmitting to the controller a signal indicative of the measured flow rate, and (iii) comparing the measured flow rate to a required flow rate for the respective formulation or one or more formulation components.

In some embodiments, the identifiers are on plural component containers and each component container contains one or more respective formulation components sealed with respect to ambient atmosphere in the component container. Some embodiments further comprise (i) reading electronic identifiers of plural component containers, (ii) transmitting read electronic identification data to the controller, (iii) comparing via the controller the read electronic identification data to required identification data for a respective formulation, and (iv) transmitting via the controller a signal to proceed if the read electronic identification data substantially matches the required identification data for a respective formulation.

Some embodiments further comprise (i) reading electronic identifiers of plural component containers, plural sterile connectors, and at least one formulation container, (ii) transmitting read electronic identification data to the controller, (iii) comparing via the controller the read electronic identification data to required identification data for a respective formulation, and (iv) transmitting via the controller a signal to proceed if the read electronic identification data substantially matches the required identification data for a respective formulation.

Some embodiments further comprise (i) reading electronic identifiers on each of a formulation container and one or more closed sterile transfer connector assemblies, (ii) determining based on the read identification information whether the formulation containers and sterile connectors are correctly connected, and (iii) based on the determination of step (ii), proceeding or not to direct or otherwise flow closed sterile transfer formulation components to the formulation container through the closed sterile transfer connector assemblies.

Some embodiments further comprise (i) introducing a plurality of primary devices and process devices into a formulation enclosure, wherein the devices are sealed and empty; (ii) upon or during passage into the formulation enclosure, reading electronic identifiers on at least a plurality of such devices; (iii) determining with the controller if any such device was not sterilized but should have been sterilized based on the read identification information; and (iv) generating a signal indicating if any such device was not sterilized. In some such embodiments, the process devices include sterile connector assemblies and the primary devices include formulation component containers. The method further includes (i) connecting the formulation component containers to a formulation container with the sterile connector assemblies; (ii) placing each of a plurality of connected sterile connectors in respective connector supports; (iii) reading with a sensor on each connector support the identification information of the respective connector in the support; (iv) transmitting read connector identification information to the controller; and (v) comparing the read connector information to required connector information.

In accordance with another aspect, an apparatus comprises (i) a plurality of primary devices or process devices, wherein each device is sealed, empty and includes an electronic identifier; (ii) one or more of a formulation enclosure or a filling enclosure, wherein each enclosure includes a door for the passage of one or more primary devices or process devices into and/or out of the enclosure; (iii) a scanner configured to read the electronic identifiers prior to, during or upon passage through the door, and transmitting the read identification information; and (iv) a controller configured to receive the read identification information from the scanner, compare the read identification information to required identification information for a respective specification, and transmit a signal to further proceed with a process in the enclosure or not based on the comparison.

In some embodiments, the enclosure is a formulation enclosure, the primary devices include plural component containers and at least one formulation container, and the process devices include plural sterile connectors. In some embodiments, the enclosure is a filling enclosure, the primary devices include plural dispensing devices or containers, and the process devices include filling kits. In some embodiments, each filling kit includes a conduit, a sterile connector located at one end of the conduit, and a filling head located at another end of the conduit. The sterile connector is configured to transfer substance by sterile closed transfer into the conduit and to a filling head, and the filling head is configured to transfer by closed sterile transfer the substance from the conduit into the dispensing devices or containers.

One advantage of the methods and apparatus of the present disclosure is that the primary devices can be closed, and thus formed with closed, empty, product-receiving chambers, at inception, such as when formed in a mold. Another advantage is that the product-receiving chambers of such primary devices can be sterile, or near sterile, particle free and/or pyrogen free, at inception, such as when formed in a mold. Another advantage is that the empty devices can be sterilized, such as by subjecting the devices to radiation, for example, gamma or ebeam radiation, if desired, to ensure sterility of the closed, empty, product-receiving chambers. Yet another advantage is that, in some cases, the closed, empty, product-receiving chambers are substantially pyrogen free and substantially particle free.

Another advantage of the methods and apparatus of the present disclosure is that each of a plurality of primary devices and each of a plurality of process devices includes an electronic identifier that identifies and distinguishes the respective device from other devices. Yet another advantage is that the methods and apparatus trace the primary and process devices by scanning or otherwise reading their electronic identifiers at each requisite stage of processing, and transmitting such read information to the controller. The controller stores such read identification information at each requisite stage of processing, such as in an associated database, to thereby trace each device through its processing and to record the processing. Based on the stored information, the controller confirms whether or not each such device has been subjected to the requisite prior processing for the respective stage. If any such device has not been subjected to the requisite prior processing for a respective stage, the controller flags the device, and may prevent the respective processing stage from proceeding for the flagged device, or otherwise prevent the processing from further proceeding until the error is corrected. For example, if the devices require sterilization prior to a respective stage of processing, such as a formulation or filling stage, and if the database indicates that the device was not previously sterilized, the controller flags the respective device to prevent the non-sterilized device from being used in a formulation or fill process.

Another advantage is that the methods and apparatus can scan or otherwise read the electronic identifiers at or about the time of entry of each such device into a processing enclosure, such as a formulation (or compounding) enclosure for formulating (or compounding) a product by closed sterile transfer, or a filling enclosure for filling a product into primary devices by closed sterile transfer. The scanned or otherwise read electronic identifier information is transmitted to the controller which can, in turn, confirm whether or not each such device has been subjected to all requisite prior processing, and can confirm whether or not all requisite primary and process devices are introduced into an enclosure for performing the respective process, such as in accordance with a customer or other specification. For example, the electronic identifiers can be read to ensure that each primary device is sterilized closed prior to introduction into a formulation or filling enclosure. Yet another advantage is that the controller can determinate based on the read electronic identifier information whether the process devices are connected to the correct process devices, such as whether the correct formulation or formulation component containers are connected to the correct sterile connector assemblies, whether the correct filling kits are connected to the correct formulation container(s), and/or whether the correct primary containers, such as vials or other dispensing devices, are sterile filled with a formulation in the sterile filling enclosure. Preferably, the electronic identifiers are read at each stage of production, such as throughout formulation and/or fill processing, to trace the primary devices throughout their processing, confirm that each such device was subjected to all requisite processing prior to performing each respective stage of processing, and to ensure that the filled-finished products are correctly processed. Each station throughout the requisite processing of a primary device reads the respective electronic identifier and transmits the read information to the controller that, in turn, stores the information in a database to trace and record the processing of the device, and ensure that the device is correctly processed.

Another advantage of the methods and apparatus is that the sterile connector assemblies can be mounted in supports that can confirm that the correct sterile connector assembly is mounted in each support, and the supports can sense and transmit to the controller the flow rate of substance through the respective sterile connector assembly or an associated conduit. The controller can trace and record the flow rate information for a respective formulation component or formulation and, in turn, control the respective pump(s) through feedback control to ensure that each flow rate is maintained at a predetermined level or otherwise in accordance with a respective specification. As a result, the methods and apparatus can digitally control the relative ratio of formulation components based on their relative flow rates into a mixing chamber to thereby control the final formulation and the concentration of ingredients therein. Yet another advantage is that the controller can monitor and control the identity of the ingredients to be mixed, their sequential order of mixing and/or their relative flow rates into the mixing chamber, to control the relative proportions of ingredients in the formulation, and the residence time of mixing or of location in the mixing chamber. Another advantage is that the methods and apparatus can thereby ensure that each final formulation is produced in accordance with a respective product specification on a consistent basis from one lot to the next and/or from one product site to the next, that the primary devices are sterile, and/or that the formulation or other product filled into the primary devices are sterile.

Yet another advantage is the methods and apparatus fill the formulation or other product into the primary devices by closed sterile transfer where, for example, the closed filling needle does not open until after the needle eye(s) penetrate through the elastic septum of the primary device, and thereby ensures that the formulation or other product is transferred from the filling head into the sealed, sterile, empty product-receiving chamber of the primary device by closed sterile transfer. Yet another advantage is that the formulation or other sterile product is never exposed to or in contact with the ambient environment throughout its processing from device manufacture, to formulation, to filling. Yet another advantage is that the sterile product may be sealed and prevented from exposure to the ambient atmosphere up until injection or other form of delivery to a patient.

Yet another advantage is that the methods and apparatus can ensure that (i) the primary devices to be filled with sterile formulations are empty and sterile, (ii) the process devices that are used to closed sterile transfer the sterile formulations between or into primary devices are sterile, and (iii) the transfer of sterile formulations between process and primary devices is by closed sterile transfer and therefore maintains the sterility of the product and prevents exposure of the product to the ambient atmosphere during transfer. The sterile connector assemblies and filling heads of the methods and apparatus prevent exposure of the transferred substance to the ambient environment during transfer from a process device to a primary device, and therefore ensure that the sterile substance remains sterile throughout such transfers. As a result, the method and apparatus ensure that the filled-finished primary devices contain sealed, sterile products or substances within their storage chambers. Yet another advantage is that such storage chambers, and the sterile products or substances contained within them, can be not only sterile, but also pyrogen free. For example, the primary devices can be closed in their molds to ensure that the closed, empty storage chambers are pyrogen free. The critical surfaces of the primary devices, such as product vials or other containers, are not exposed to an overpressure of air from a HEPA filter, let alone to the ambient environment, but rather are sealed within the closed primary devices. Similarly, the critical surfaces of the process devices (i.e., the interior surfaces that can contact the product or substance transferred therethrough), such as the formulation kits and filling kits, and their associated sterile connectors and filling heads, are neither exposed to the overpressure of air from a HEPA filter or to the ambient environment, but rather are sealed within the closed process devices.

Yet another advantage of the methods and apparatus of the present disclosure is that a regulatory auditor may rely on the scanned electronic identifier information, and the associated database tracing the processing of each such device by scanning or otherwise reading the electronic identifiers at each requisite stage of processing, to confirm that each primary device and process device has been correctly processed in accordance with regulatory requirements. Each device can be electronically traced throughout its processing from inception, such as molding of the device, to compounding the formulation by closed sterile transfer, to filling the formulation into primary devices by closed sterile transfer, to final packaging and labeling to create a filled-finished product. The regulatory auditor can review the recorded electronic identifier information to confirm that each primary device was correctly processed at each requisite stage of its processing, and that each process device used in each such stage was correctly processed. Yet another advantage of the methods and apparatus is that they allow for remote access to the controller and/or its database, such as through a wireless internet or other connection, to remotely monitor the read electronic identifier information, or otherwise access the recorded information in the database, to audit the information.

Accordingly, an advantage of the methods and apparatus is that they can significantly reduce the risk of contamination of products compared to previously known methods and apparatus throughout formulating, filling, storage and dispensing processes.

Another advantage is that they can do so at increased speed. Yet a further advantage is that they can do so at significantly reduced costs. Yet another advantage is that they allow individuals, companies and governmental authorities to respond quickly to product shortages, and also do so with a “just in time” response.

A further advantage is that they address problems with previously-known technology, including lack of sterility, lack of compliance with Good Manufacturing Practices (GMPs), lack of product quality, lack of product consistency, and presence of undesirable particles and foreign objects in products.

Other objects and advantages of the methods and apparatus of the present disclosure will become more readily apparent in view of the following detailed description of embodiments and accompanying drawings.

### BRIEF DESCRIPTION OF THE DRAWINGS

FIG. 1 is a schematic illustration of a formulation and filling apparatus including engineered traceability for tracking components, including the primary and process containers or other devices, and controlling the processing thereof, in accordance with an embodiment of the invention;

FIG. 2 is a perspective view of the formulation and filling apparatus of FIG. 1;

FIG. 3 is a perspective view of the formulation vessel of FIG. 1;

FIG. 4 is a cross-sectional view of an embodiment of a sterile connector assembly of FIG. 1 illustrating the sterile connector assembly in a disconnected condition;

FIG. 5 is a perspective view of an embodiment of filling kits of FIG. 1;

FIG. 6 is a perspective view of another embodiment of filling kits of FIG. 1;

FIG. 7A is an elevational view of the sampling valve of FIG. 1;

FIG. 7B is a cross-sectional view of the sampling valve of FIG. 7A;

FIG. 8 is a top perspective view of an embodiment of a sterile connector support or cradle of FIG. 1 with the cover of the cradle in an open position;

FIG. 9 is a top perspective view of the cradle of FIG. 8 with a sterile connector located therein;

FIG. 10 is a partial cross-sectional, perspective view of a filling needle assembly of the filling apparatus of FIG. 1 illustrating the filling needle penetrating an elastic septum of a pouch;

FIG. 11 is a perspective view of a pouch that may be filled in the filling apparatus of FIG. 1;

FIG. 12 is a perspective view of the filling apparatus of FIG. 1;

FIG. 13 is a depiction of components of the apparatus of FIG. 1 and exemplary closed devices that can be filled by the apparatus of FIG. 1;

FIG. 14A is a schematic illustration of the procedural steps involved in initiating a customer recipe for closed sterile transfer compounding/formulation and filling and electronically tracing such steps;

FIG. 14B is a schematic illustration of the procedural steps involved in transferring a release inventory for closed sterile transfer formulation of a compound and electronically tracing such steps;

FIG. 14C is a schematic illustration of the procedural steps involved in transferring the release inventory of FIG. 14B into the compounding/formulation enclosure and electronically tracing such steps;

FIG. 14D is a schematic illustration of the procedural steps involved in tracing entry of the compounding, filling and other authorized personnel into the compounding/formulation and filling enclosures;

FIG. 14E is a schematic illustration of the procedural steps involved in closed sterile transfer compounding/formulation of a product and electronically tracing such steps;

FIG. 14F is a schematic illustration of the procedural steps involved in setting up the closed sterile transfer filling assembly for performing a closed sterile transfer filling process and electronically tracing such steps; and

FIG. 14G is a schematic illustration of the procedural steps involved in a closed sterile transfer filling process and electronically tracing such steps.

### DETAILED DESCRIPTION OF EMBODIMENTS OF THE INVENTION

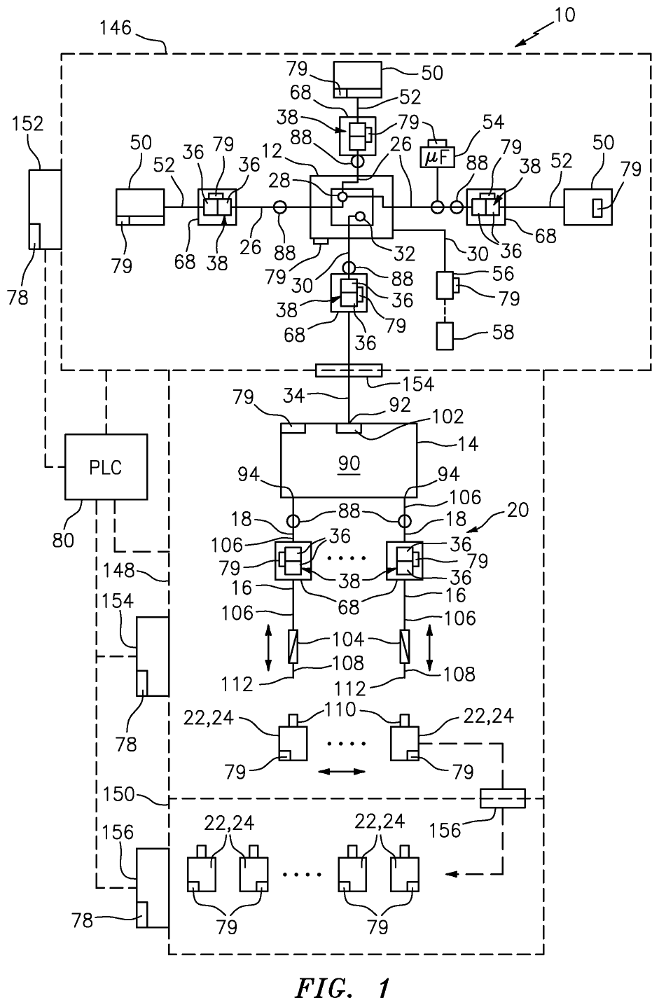

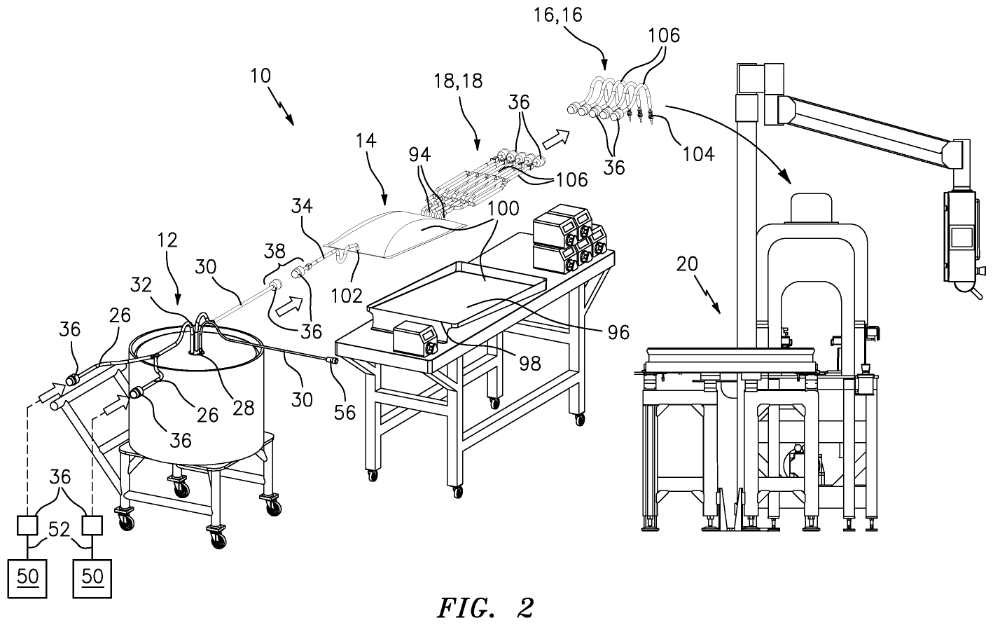

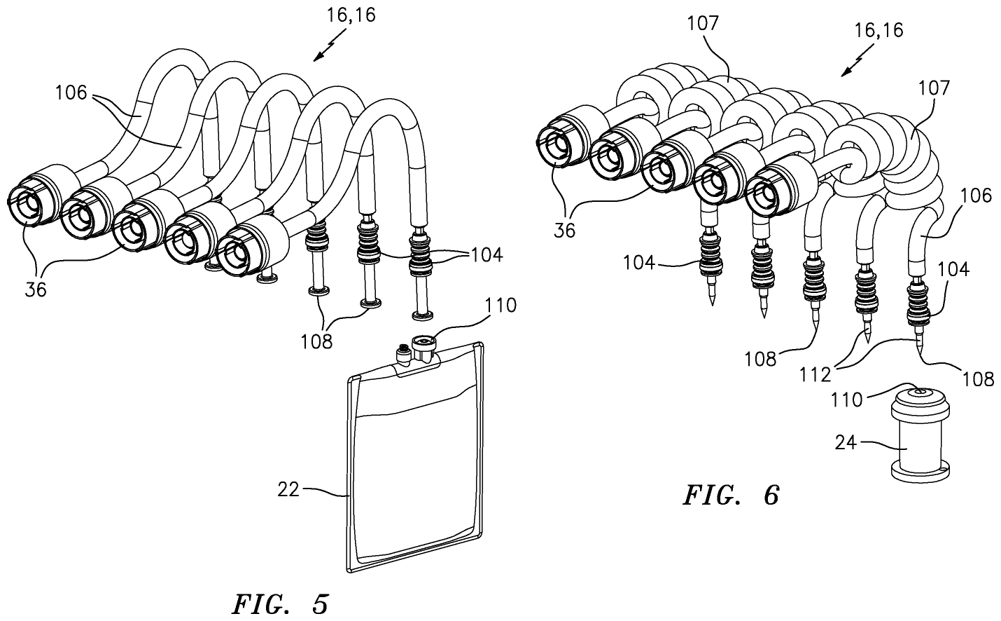

In FIGS. 1 and 2, an apparatus in accordance with an embodiment of the present invention is indicated generally by the reference numeral 10. The apparatus 10 comprises a closed sterile transfer formulation container or tank 12 for creating therein sterile formulations, a closed sterile transfer intermediate or surge tank 14 for receiving the sterile formulation by closed sterile transfer from the formulation tank, and a plurality of closed sterile transfer filling kits 16, 16 for receiving the sterile formulation by closed sterile transfer from the surge tank 14. A plurality of closed sterile transfer pump kits 18, 18 are connectible in fluid communication between the surge tank 14 and the respective closed sterile transfer filling kits 16, 16 for pumping the sterile formulation by closed sterile transfer from the surge tank 14 to the respective closed sterile transfer filling kits. The filling kits 16, 16 are mounted in a filling assembly 20 for filling the sterile formulation by closed sterile transfer from the filling kits to respective dispensing or delivery containers, shown typically at 22 or 24 in FIGS. 5 and 6, respectively.

The term “closed sterile transfer” or “closed transfer” means that the fluid or other substance, such as one or more formulation components or a formulation, is transferred without exposure of the transferred substance to the ambient atmosphere, and the transferred substance is sealed with respect to ambient atmosphere throughout the transfer. In the illustrated embodiments, the term “closed sterile transfer” or “closed transfer” further means transferring a sterile substance without exposure of the substance to germs or other contaminants to thereby maintain the substance sterile throughout the transfer. A “primary” container or device is a container or device that receives or holds one or more formulation ingredients or formulations. A “process” container or device is a container or device used to process or used in the processing of one or more formulation ingredients or formulations, and that is not a primary container or device. With reference to FIGS. 1 and 2, the formulation tank 12, surge tank 14 and dispensing or delivery containers 22, 24, are primary containers or devices, whereas the filling kits 16, 16, and transfer pump kits 18, 18 are process containers or devices. Additional primary and process containers and devices are described below.

A plurality of inlet closed sterile transfer assemblies 26, 26 are connectible in fluid communication by closed sterile transfer to an inlet port 28 of the formulation tank 12 for introducing sterile formulation components, such as ingredients or groups of ingredients, into the formulation tank, and mixing or otherwise making a sterile formulation therein. An outlet closed sterile transfer assembly 30 is connectible in fluid communication by closed sterile transfer between an outlet 32 of the formulation tank 12 and the surge tank 14. The surge tank 14 also includes an inlet closed sterile transfer assembly 34 that is connectible in fluid communication between the outlet closed sterile transfer assembly 30 of the formulation tank 12 and the surge tank 14 for transferring the sterile formulation from the formulation tank to the surge tank.

The apparatus 10 includes a plurality of sterile connectors 36, 36 for effecting the closed sterile transfer of substance between the various components of the apparatus. Each sterile connector 36 defines a disconnected condition and a connected condition. In the disconnected condition, each sterile connector is closed and the interior of the sterile connector is hermetically sealed with respect to the ambient atmosphere. Thus, in the disconnected condition, each sterile connector maintains the interiors of the components that it is connected in fluid communication with, hermetically sealed with respect to the ambient atmosphere. In the connected condition, each sterile connector 36 is connected in fluid communication to another sterile connector 36 to form a respective sterile connector assembly 38, and each connector assembly 38 defines a closed sterile conduit extending in fluid communication through the sterile connector for the closed sterile transfer of substance therethrough. The closed sterile conduit of each connector assembly 38 is hermetically sealed with respect to the ambient atmosphere, and is sterile to thereby prevent the exposure of substance transferred therethrough to the ambient atmosphere and to maintain such substance sterile throughout the transfer.

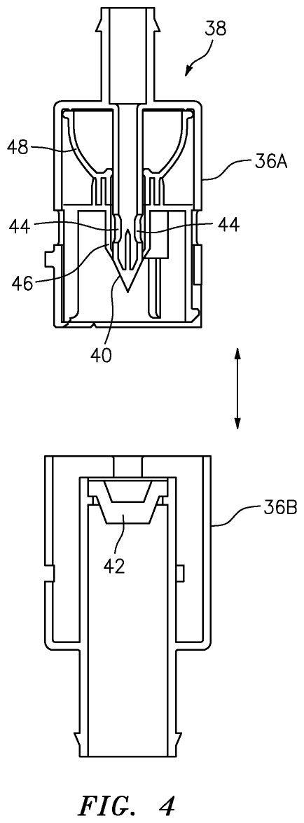

As shown typically in FIG. 4, in the illustrated embodiment, each sterile connector assembly 38 comprises a male connector 36A including a piercing member 40, and a female connector 36B including an elastic septum 42. As indicated by the arrows in FIG. 4, the male and/or female connectors 36A, 36B are movable relative to each other between disengaged and engaged positions, during movement between the disconnected and connected positions. During connection, the piercing member 40 of the male connector 36A penetrates the elastic septum 42 of the female connector 36B, and the elastic septum decontaminates the piercing member by physical interaction therewith. In the connected position, the transferred substance, such as sterile formulation components or formulation, are flowable through the sterile connector assembly 38 and sealed therein with respect to the ambient atmosphere.

The piercing member 40 of each male connector 36A includes outflow apertures 44, 44 and a closure 46 movable between a closed position covering the outflow apertures (as shown), and an open position exposing the outflow apertures (not shown). The closure 46 is normally biased into the closed position by a spring 48, such as the illustrated elastic, dome-shaped spring, a coil spring, or any other type of spring that is currently known or later becomes known. The closure 46 defines a locked condition and an unlocked condition. The closure 46 is in the locked condition prior to and during penetration of the elastic septum 42. Then, after the outflow apertures 44, 44 of the piercing member 40 penetrate the septum 42, the closure 46 is unlocked to allow further movement of the piercing member relative to the closure to, in turn, expose the outflow apertures and allow the flow of sterile substance, such as one or more formulation components or formulations, therethrough.

As may be recognized by those of ordinary skill in the pertinent art based on the teachings herein, the sterile connectors may take the form of any of numerous different sterile connectors that are currently known, or that later become known. Examples of sterile connectors suitable for use in the present invention are disclosed in the following patents and patent applications, the disclosures of which are hereby incorporated by reference in their entireties as part of the present disclosure: U.S. Pat. No. 8,671,964, issued Mar. 18, 2014, titled “Aseptic Connector with Deflectable Ring of Concern and Method;” U.S. patent application Ser. No. 13/874,839, filed May 1, 2013, titled “Device for Connecting or Filling and Method;” U.S. patent application Ser. No. 13/864,919, filed Apr. 17, 2013, titled “Self Closing Connector;” and U.S. patent application Ser. No. 14/536,566, filed Nov. 7, 2014, titled “Device for Connecting or Filling and Method.” As should be appreciated by those of ordinary skill in the art, other suitable sterile connectors that are either known or subsequently become known also may be used.

As shown in FIGS. 1 and 2, each of the inlet closed sterile transfer assemblies 26, 26 includes a respective sterile connector 36 at its inlet end. In the illustrated embodiment, each inlet sterile connector is a female connector 36B; however, a female connector is not required, and if desired, a male connector or other type of connector may be used instead. The apparatus 10 further includes a plurality of formulation component containers or tanks 50, 50. Each formulation component container 50 includes a respective substance, such as a formulation component or plurality of formulation components, for closed sterile transfer into the formulation tank 12. Each formulation container 50 includes a respective outlet closed sterile transfer assembly 52, and each outlet closed sterile transfer assembly 52, 52 includes on its outlet end a respective sterile connector 36. The sterile connectors 36, 36 of the outlet closed sterile transfer assemblies 52, 52 are connectable to the sterile connectors 36, 36 of the inlet closed sterile transfer assemblies 26, 26 to allow for closed sterile transfer of the substances, such as formulation components, from the formulation component containers 50, 50 into the formulation tank 12.

In some embodiments of the present invention, the formulation components may be sterilized, such as by relatively cold sterilization or by relatively hot sterilization. As shown typically in FIG. 1, one or more of the inlet closed sterile transfer assemblies 26, 26 includes an in-line microfilter (μF) 54 for sterilizing the liquid or other fluid passing therethrough by microfiltration. The microfilter 54 may take the form of any of numerous different sterilizing filters that are currently known, or that later become known. Alternatively, the liquid may be sterilized by another relatively cold sterilization technique, such as by subjecting the liquid to a sterilizing ebeam radiation. Relatively cold sterilization may be preferred for formulation components, such as noble ingredients, that can be degraded or otherwise damaged by relatively hot sterilization. Other formulation components or ingredients that can tolerate thermal or relatively hot sterilization can be separately sterilized, such as by terminal sterilization in the respective formulation component container 50. Then, the separately sterilized components can be mixed, post sterilization, in the formulation container 12.

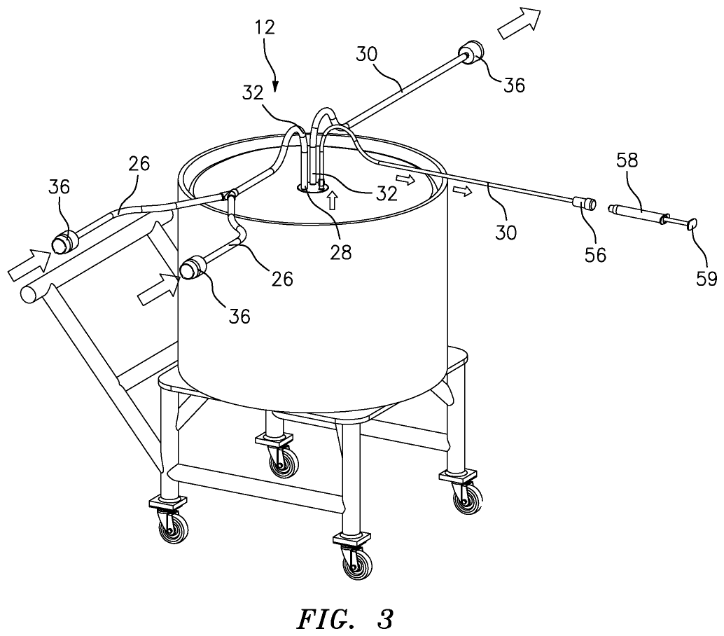

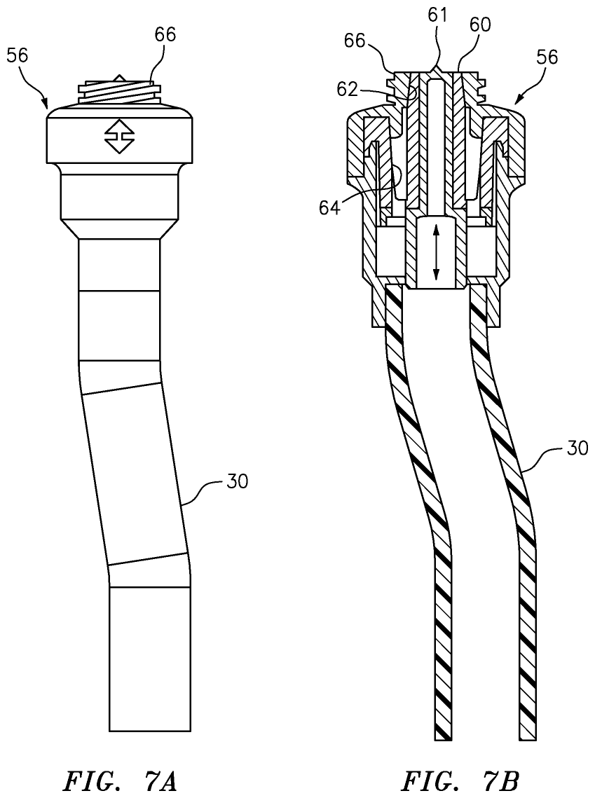

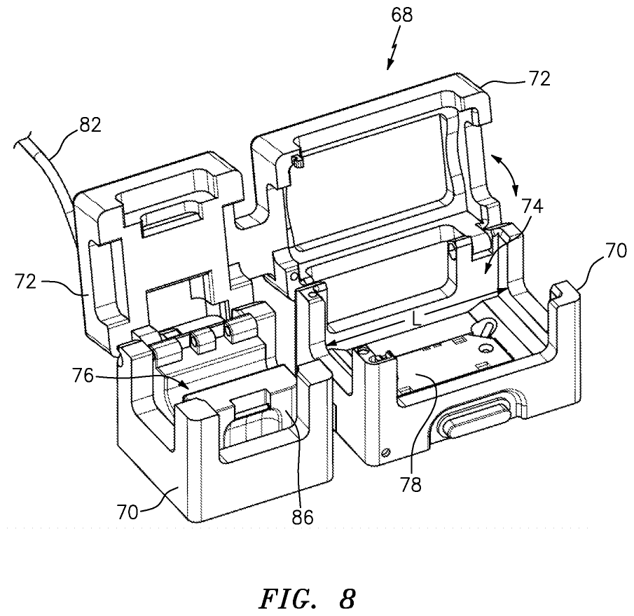

As also shown in FIGS. 1 and 3, one or more of the outlet closed sterile transfer assemblies 30 may include a formulation-sampling valve 56, such as on its distal end, and may include such a valve in lieu of a respective sterile connector. The sampling valve 56 is normally closed and seals the interior of the formulation container 12 with respect to the ambient atmosphere. A sampling device 58, such as the illustrated syringe (FIG. 3), is connectable to the sampling valve 56 to withdraw one or more samples of formulation from the formulation container 12 into the sampling device. The sampling valve 56 allows the sample to flow through the valve and into the sampling device 58 while nevertheless hermetically sealing the interior of the respective outlet closed sterile transfer assembly 30 and formulation container 12 with respect to the ambient atmosphere. With reference to FIGS. 7A and 7B, the illustrated sampling valve 56 includes an elastic, annular valve member 60, a relatively rigid, annular valve seat 62, and an elastic spring 64 that normally biases the valve member into engagement with the valve seat to form a hermetic, annular seal between the valve member and valve seat and thereby close the valve. In order to withdraw a sample through the sampling valve 56, the tip of the sampling device 58 engages a protuberance 61 at the distal end of the valve member 60 through a mechanical connection 66 on the distal end of the valve, such the illustrated luer-lock connector. As indicated by the arrows in FIG. 7B, movement of the sampling device 58 into engagement with the sampling valve 56, such as through the luer-lock connector, moves the valve member 60 inwardly and away from the valve seat 62 against the bias of the spring 64 to, in turn, open the valve. A plunger 59 of the sampling syringe 58 is then withdrawn (or pulled away from the sampling valve 56) to withdraw a sample of the formulation component(s) and/or formulation through the open valve and into the body of the syringe 58. Once the sample is withdrawn, the sampling device 58 is disengaged from the valve member 56 to allow the spring 64 to bias the valve member 60 into engagement with the valve seat 62, and thereby seal the interior of the formulation container with respect to the ambient atmosphere.

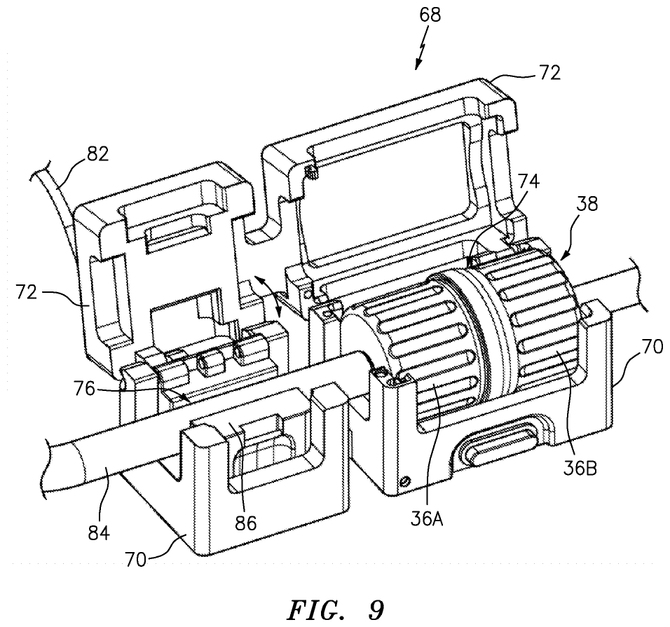

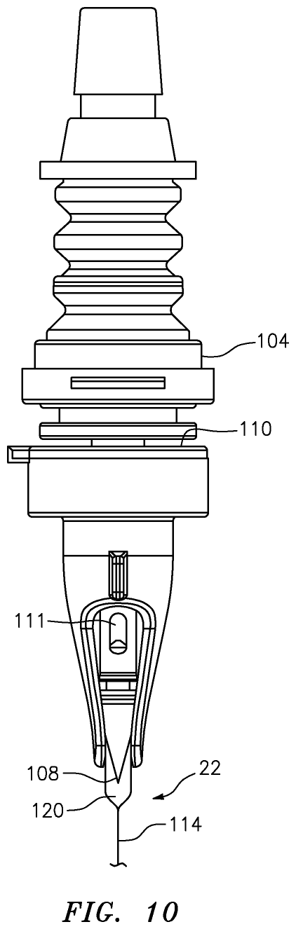

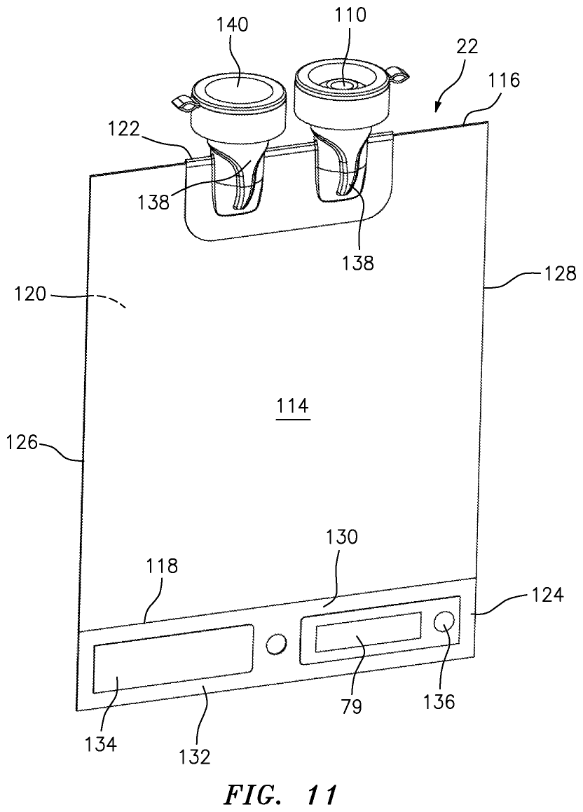

Referring to FIGS. 8 and 9, a sterile connector assembly support or cradle 68 can be utilized to help ensure that the proper connector assembly 38 is being used and/or that the connector halves 36, 36 are properly connected together. Cradle 68 has a main body 70 and a door or cover 72 which, as indicated by the arrows in FIGS. 8 and 9, is movable relative to the body 70 between an open position as shown, to a closed position (not shown) in which the cover 72 engages the main body 70. The main body 70 defines a first interior cavity 74 and a second interior cavity 76. The first interior cavity 74 is sized and shaped to receive therein a respective connector assembly 38 only when the connector halves 36, 36 are in the connected position. More specifically, the first interior cavity 74 defines a length “L” that permits the connector assembly to be received in the first cavity 74 only when the connector halves 36, 36 are properly connected together. If the connector halves 36, 36 are not properly connected together, the overall length of the connector assembly 38 will exceed the length L of the first interior cavity 74, and the connector assembly will not fit into the first interior cavity. The first interior cavity 74 also contains an electronic reader or sensor 78 configured to read respective electronic identifiers 79 on each connector assembly 38 and/or connector halves 36, 36 present in the interior cavity 74. In some embodiments, the sensor is a radio-frequency identification or “RFID” reader adapted to read corresponding electronic identifiers 79 (FIG. 1), such as RFID tag(s), present on one or more connector assemblies 38 and/or connector half or halves 36A, 36B. In other embodiments, the sensor 78 is a barcode reader that is configured to read a barcode(s) tag or label on the connector assemblies and/or connector half or halves. When the sensor 78 determines the identifying information regarding the connector assembly 38 and/or connector halves 36, 36 from the respective electronic identifier or tag 79, it transmits that information to a program logic controller (“PLC”) 80 (FIG. 1) via a wire or cable 82 or, alternatively, wirelessly, to indicate (1) that the respective connector assembly is properly connected and in position in the cradle 68, and (2) the identity of the respective connector assembly and/or the connector halves.

The second interior cavity 76 is sized and configured so as to be able to receive therein a portion of a flow channel 84 of the connector 36, e.g., a tube or conduit. The second interior cavity 76 contains therein a flow meter 86 that measures, determines and/or meters the flow rate of a substance through the connector assembly when the flow channel 84 is in the second interior cavity 76. The flow meter 86 may measure the flow rate by any existing or later-developed technology, including but not limited to optical, laser, ultrasonic, and/or magnetic technology. The flow meter 86 transmits its readings to the PLC 80 via the wire 82 or, alternatively, wirelessly. Each inlet closed sterile transfer assembly 26, 26 and each outlet closed sterile transfer assembly 30, 30 includes a respective flow channel 84 sealingly connected in fluid communication to the respective sterile connector 36. In the illustrated embodiment, each flow channel 84 is defined by a flexible tube or conduit. As shown in FIG. 1, the apparatus 10 further comprises a plurality of pumps 88, such as peristaltic pumps, that engage the exterior surfaces of respective flexible tubular conduits 84 to pump the sterile formulation components or formulation through the tubular conduits. Each peristaltic pump 88 is connected by a wire (not shown) or alternatively is connected wirelessly to the PLC 80 in order to allow the PLC to control the operation and speed of each pump. Based on the flow rates indicated by the flow meters 86, the PLC 80 can control the speed of the respective pump to achieve and/or maintain a required flow rate, to set the flow rates of respective formulation components at predetermined or otherwise desired ratios in order to obtain a predetermined or otherwise desired formulation, or otherwise as required or desired.

As seen in FIGS. 8 and 9, when the cover 72 is in the open position, the connector assembly 38 may be inserted into the cradle 68, and removed from the cradle. When the cover 72 is closed, the cover sufficiently blocks access to the interior cavities 74, 76 so that the connector assembly 38 and/or the flow channel 84 may not be removed from the cradle. Thus, the cover 72 helps prevent inadvertent removal or dislodging of the connector assembly 38 and/or flow channel 84 from the first and second cavities 74 and 76, respectively. As shown in FIG. 2, in the illustrated embodiment, each connector assembly 38 is received in a respective cradle 68; however, as may be recognized by those of ordinary skill in the pertinent art based on the teachings herein, not every connector assembly need be received in a respective cradle, if desired. The cradles and their methods of use may be the same as or similar to any of the cradles and methods disclosed in the following co-pending patent applications which are hereby incorporated by reference in their entireties as part of the present disclosure: U.S. patent application Ser. No. 15/410,758, filed Jan. 19, 2017, entitled “Devices and Methods for Formulation Processing,” which claims the benefit of U.S. Provisional Patent Application No. 62/280,696, filed Jan. 19, 2016, entitled “Formulation Processing;” and U.S. patent application Ser. No. 15/410,513, filed Jan. 19, 2017, entitled “Apparatuses and Methods for Formulating Using a Swirl Chamber,” which claims the benefit of similarly-titled U.S. Provisional Patent Application No. 62/280,691, filed Jan. 19, 2016.

As may be recognized by those of ordinary skill in the pertinent art based on the teachings herein, any of numerous different electronic identifiers and associated sensors or readers that are currently known, or that later become known, may be used in lieu of the RFID or barcode label/tag or reader/scanner. In addition, the electronic identifiers may provide any of numerous different types of information that is currently known or later becomes known, including without limitation, identification of the respective primary or process containers or devices, a condition of the respective primary or process containers or devices, such as whether the respective containers or devices have been subjected to a sterilization process, i.e., whether they were sterilized, such as by gamma, ebeam or other sterilization process, and the stage of processing of the respective containers or devices, such as the status of the formulation or filling processing of the respective containers or devices. The PLC 80 similarly may take the form of any of numerous different programmable or other electronic controllers or other computerized devices that are currently known or later become known, the apparatus 10 may include any desired number of such controllers or computerized devices, and the controller(s) and/or computerized devices may be connected to any desired number of other computers or computer networks, in any of numerous different ways, that are currently known, or later become known. In addition, each PLC 80 or other computerized device may include software for tracking and monitoring the primary and process containers or devices in any of numerous different ways that are currently known or later become known.

In one embodiment, the PLC 80 traces each primary and process container or device through the respective electronic identifier attached or otherwise associated with each such container or device. Through such electronic identification and tracing, the PLC 80 monitors and confirms that the required primary and process containers are present, and where applicable, are connected to each other as required, for each step of the formulation, filling and/or other processes. For example, the PLC 80 can trace each sealed, empty primary or process container or device through sterilization, such as gamma or ebeam sterilization, via its respective electronic identifier, to confirm that each such container or device is sterilized prior to subjecting the respective container or device to further formulation or fill processing. If, on the other hand, the PLC 80 detects that a primary or process container or device that was not subject to sterilization is presented for formulation or fill processing, the PLC can generate an alarm to prevent further processing of the unsterilized container or device, or otherwise flag the respective container or device for rejection. Similarly, the PLC 80 can trace and confirm that each primary or process container or device is subjected to each requisite step or stage of its respective processing by reading the respective electronic identifier at each such step or stage, and if any such container or device is presented to or otherwise introduced for processing at a respective step or stage without having completed all requisite steps or stages required prior to the respective step or stage, the PLC 80 can generate an alarm or otherwise flag the respective container or device for inspection or rejection. The PLC 80 stores the read identification information at each requisite stage of processing in one or more associated databases to trace each device through its processing and to record the processing. Based on the information stored in the database, the PLC 80 confirms whether or not each such device has been subjected to the requisite prior processing for the respective stage. The PLC 80 and/or its associated database(s) can be remotely accessed, such as through a wireless internet or other connection, to remotely monitor the read electronic identifier information, or otherwise access the recorded information in the database, such as to audit the information.

As shown in FIG. 3, the formulation container 12 may take the form of a flexible bag, and the formulation components may be mixed by rotating an impeller (not shown) within the bag. The impeller may be magnetically suspended within the interior of the bag, and the impeller may be rotatably driven while suspended within the bag to facilitate mixing of the formulation components within the bag. The magnetic drive unit (not shown) is connected by a wire or wirelessly to the PLC 80 to allow the PLC to control the operation and speed of the impeller to, in turn, control mixing of the formulation components within the bag.

As shown in FIGS. 1 and 2, the surge tank 14 defines an intermediate formulation chamber 90 that is sealed with respect to ambient atmosphere, an inlet port 92 in communication with the intermediate formulation chamber, and a plurality of outlet ports 94, 94 in fluid communication with the intermediate formulation chamber. The inlet closed sterile transfer assembly 34 is sealingly connected in fluid communication with the inlet port 92. A respective closed sterile transfer pump kit 18 is sealingly connected to each outlet port 94. The sterile connector 36 of each sterile transfer pump kit 18 and of the inlet closed sterile transfer assembly 34 is normally closed and seals the intermediate formulation chamber 90 with respect to ambient atmosphere. As shown in FIG. 1, each such sterile connector 36 is engageable with another sterile connector 36 and connectable in fluid communication therewith to form a respective sterile connector assembly 38 in order to (i) allow closed sterile transfer of formulation through the sterile connector and into the intermediate formulation chamber 90, or (ii) allow closed sterile transfer of formulation out of the intermediate formulation chamber 90 and through the sterile connector. In each case, the closed sterile transfer is without exposure of the formulation to the ambient atmosphere, and the formulation is sealed with respect to ambient atmosphere.

As shown in FIG. 2, the surge tank 14 is seated on a surge tank support 96. The surge tank support 96 is oriented at an acute angle with respect to a horizontal plane defining an upper end 98 and a lower end 100. The outlet ports 94, 94 of the surge tank 14 are located at the lower end 100 of the support to direct the formulation within the intermediate formulation chamber 90 toward the lower end 100 and outlets 94, 94. The surge tank 14 also includes a venting valve 102 that allows gas to vent out of the intermediate formulation chamber 90 but prevents gas from flowing into the intermediate formulation chamber. The venting valve 102 is located at or adjacent to the upper end 98 of the support 96.

As shown in FIG. 1, the closed sterile transfer filling assembly 20 is connectable in sterile fluid communication with the formulation chamber of the formulation container 12 or the intermediate formulation chamber 90 of the surge tank 14. In the illustrated embodiment, the closed transfer filling assembly 20 includes a plurality of closed transfer filling devices 104, 104. Each closed transfer filling device 104 is sealingly connected at the distal end of a respective closed sterile transfer filling kit 16. Each closed transfer filling device 104 includes a respective valve or closure movable between a closed position sealing the interior of the closed transfer filling device and any formulation therein from the ambient atmosphere, and an open position allowing the flow of formulation therethrough.

As indicated by the arrows in FIG. 1, each filling device 104 is movable between a first position and a second position. In the first position, each filling device 104 is locked in a closed position sealing any formulation therein from the ambient atmosphere. In the second position, each filling device 104 is engageable with a respective primary container or device, such as the dispensing containers 22, 24. In the second position, the valve or closure of each filling device 104 is movable to the open position and the filling device is connectable in sterile fluid communication with the interior of the primary container or device, such as the dispensing containers 22, 24, to allow the flow of sterile formulation through the filling device and into the interior of the dispensing container.

As shown in FIGS. 5 and 6, each closed sterile transfer filling kit 16 includes a flexible tubular conduit 106 sealingly connected in fluid communication with the respective closed transfer filling device 104. As shown in FIG. 2, each closed sterile transfer pump kit 18 also includes a flexible tubular conduit 106 sealingly connected in fluid communication with the respective sterile connector 36. The exterior surface of each flexible tubular conduit 106, 106 is engageable by a peristaltic pump, such as one of the peristaltic pumps 88 (FIG. 1), for pumping the sterile formulation with the peristaltic pump through the tubular conduit and, in turn, through the pump kit or filling kit, respectively. As shown in FIG. 6, the flexible tubular conduit 106 of each closed sterile transfer filling kit 16, 16 may include a helicoidal portion 107. In the illustrated embodiment, each helicoidal portion 107 is spaced between the sterile connector 36 on the inlet end of the respective filling kit 16, and the closed transfer filling device 104 on the outlet end of the respective filling kit. Accordingly, each flexible tubular conduit 106 defines a portion of the conduit on the inlet and outlet ends of the helicoidal portion that is not helicoidal. Each helicoidal portion 107 defines a plurality of turns. In the illustrated embodiments, each helicoidal portion 107 defines at least about 3 turns, preferably at least about 4 turns, and more preferably at least about 5 turns. One such helicoidal portion 107 defines about 7 turns. One advantage of the helicoidal portion is that it can facilitate precise engagement by the respective closed transfer filling device 104 with each penetrable septum of each primary container or device and/or facilitate constant fill dosing from one primary container or device to the next.

Each closed transfer filling device 104 comprises a piercing member 108, and each primary container or device, such as the dispensing containers 22, 24, includes a penetrable and resealable, elastic septum 110. In the second position of each filling device 104, the piercing member 108 is engageable with the elastic septum 110 of a respective primary container or device, such as a dispensing container. During movement between the first and second positions, the piercing member 108 penetrates the elastic septum 110 and decontaminates the piercing member by physical interaction with the elastic septum, and the formulation is sterile transferred through the piercing member and into the respective primary container or device. Each piercing member 108 includes one or more outflow apertures 111 (FIG. 10) and a closure 112. Each piercing member 108 and/or respective closure 112 is movable between a closed position where the closure covers the outflow apertures 111, and an open position exposing the outflow apertures. In the first position of each closed sterile transfer filling device 104, the closure 112 is locked in the closed position until the outflow apertures penetrate the respective septum 110. As shown typically in FIG. 10, in the second position (after the outflow apertures 111 penetrate through the respective septum 110), the closure 112 is unlocked with respect to the piercing member 108 and the closure and/or piercing member is movable relative to the other to expose the outflow apertures 111 and allow the flow of formulation therethrough and into the interior of the primary or dispensing container 22, 24. Each closed transfer filling kit 16, 16 includes a closed transfer filling device or head 104 on its distal end, a sterile connector 36 on its opposite end, and a flexible conduit 106 sealingly connected between the respective closed transfer filling head and sterile connector. The sterile connector 36 of each closed transfer filling kit 16, 16 is connectable to a sterile connector of a respective closed transfer pump kit 18, 18 that, in turn, is sealingly connected in fluid communication with the intermediate formulation chamber 90 of the surge tank 14 for the closed sterile transfer of formulation therethrough.

The closed sterile transfer filling assembly 20, closed sterile transfer filling devices, and methods of closed transfer filling, may take the form of any of the assemblies, devices or methods disclosed in the following patents and patent applications, which are hereby incorporated by reference in their entireties as part of the present disclosure: U.S. patent application Ser. No. 15/434,468, filed Feb. 16, 2017, entitled “Controlled Non-Classified Filling Device and Method,” which is a divisional application of similarly-titled U.S. patent application Ser. No. 14/214,890, filed Mar. 15, 2014, now U.S. Pat. No. 9,604,740, which, in turn, claims the benefit of similarly-titled U.S. Provisional Patent Application No. 61/798,210, filed Mar. 15, 2013; U.S. patent application Ser. No. 15/267,131, filed Sep. 15, 2016, entitled “Septum That Decontaminates by Interaction With Penetrating Element,” which claims the benefit of similarly-titled U.S. Provisional Patent Application No. 62/219,035, Sep. 15, 2015; U.S. Design patent application Ser. No. 29/539,571, filed Sep. 15, 2015, entitled “Septum;” U.S. patent application Ser. No. 13/450,306, filed Apr. 18, 2012, entitled “Needle With Closure and Method,” which claims the benefit of U.S. Provisional Patent Application No. 61/476,523, filed Apr. 18, 2011, entitled “Filling Needle and Method;” U.S. patent application Ser. No. 13/864,919, filed Apr. 17, 2013, entitled “Self Closing Connector,” which claims the benefit of similarly-titled U.S. Provisional Patent Application No. 61/784,764, filed Mar. 14, 2013, similarly-titled U.S. Provisional Patent Application No. 61/635,258, filed Apr. 18, 2012, and similarly-titled U.S. Provisional Patent Application No. 61/625,663, filed Apr. 17, 2012; U.S. patent application Ser. No. 14/536,566, filed Nov. 7, 2014, entitled “Device for Connecting or Filling and Method,” which is a continuation-in-part of similarly-titled U.S. patent application Ser. No. 13/874,839, filed May 1, 2013, which, in turn, claims the benefit of similarly-titled U.S. Provisional Patent Application No. 61/794,255, filed Mar. 15, 2013, and similarly-titled U.S. Provisional Patent Application No. 61/641,248, filed May 1, 2012; U.S. patent application Ser. No. 14/636,954, filed Mar. 3, 2015, entitled “Modular Filling Apparatus and Method,” which is a divisional application of similarly-titled U.S. patent application Ser. No. 13/861,502, filed Apr. 12, 2013, now U.S. Pat. No. 8,966,866, which, in turn, claims the benefit of similarly-titled U.S. Provisional Patent Application No. 61/686,867, filed Apr. 13, 2012; U.S. patent application Ser. No. 14/708,196, filed May 9, 2015, entitled “Self Closing and Opening Filling Needle, Needle Holder, Filler and Method,” which claims the benefit of similarly-titled U.S. Provisional Patent Application No. 61/991,561, filed May 11, 2014, and similarly-titled U.S. Provisional Patent Application No. 61/991,467, filed May 10, 2014; U.S. patent application Ser. No. 13/529,951, filed Jun. 21, 2012, entitled “Fluid Sterilant Injection Sterilization Device and Method,” which claims the benefit of U.S. Provisional Patent Application No. 61/499,626, filed Jun. 21, 2011, entitled “Nitric Oxide Injection Sterilization Device and Method;” and U.S. patent application Ser. No. 13/917,562, filed Jun. 13, 2013, entitled “Device With Penetrable Septum, Filling Needle and Penetrable Closure, and Related Method,” which claims the benefit of similarly-titled U.S. Provisional Patent Application No. 61/799,744, filed Mar. 15, 2013, and similarly-titled U.S. Provisional Patent Application No. 61/659,382, filed Jun. 13, 2012.

In FIG. 11, an illustrated primary container or device 22 is in the form of a pouch. The pouch 22 comprises a tubular film 114 including an inner surface and an outer surface, a first end edge portion 116 extending from approximately one side of the pouch to another side of the pouch, and a second end edge portion 118 located on an opposite end of the pouch relative to the first end edge portion, and extending from approximately one side of the pouch to another side of the pouch. A sealed interior chamber 120 is defined between opposing inner surfaces of the tubular film 114. In the illustrated embodiment, the interior chamber 120 extends from the first end 116 to the second end 118, and from one side to another side of the pouch. The pouch 22 defines a pouch aperture (not shown) in fluid communication with the interior chamber 120, and extending through the tubular film and/or between opposing edge portions of the pouch. A fitment 122 is over-molded or otherwise fixedly secured and sealed to the outer surface of the pouch along a periphery of the pouch aperture. In the illustrated embodiment, the outer surface of the tubular film 114 is at least partially melted and thermally bonded to the fitment 122 to thereby form a fluid-tight seal between the pouch and fitment about the pouch aperture.

The sealed, empty pouch 22 can be made in accordance with the following method: (i) molding a tubular film 114 including an inner surface and an outer surface, and blowing or otherwise directing micro-filtered air and/or other gas (which may be heated) through the hot tubular film during molding; (ii) flattening the molded tubular film 114; (iii) sealing the flattened tubular film at spaced locations, cutting the sealed film at the spaced locations, and thereby forming one or more empty pouches; (iv) over-molding the fitment 122 to the outer surface of each of one or more such empty pouches; and (v) preventing the collection of particles on the inner surfaces of the pouch, and the exposure of such surface to the ambient atmosphere throughout steps (i) through (iv). When formed in accordance with this method, the interior chamber 120 of the pouch is sealed, empty and sterile, and thus ready to be sterile filled by closed sterile transfer in the filling assembly 20. One advantage of the foregoing apparatus and method, is that the interior of the pouch, including the critical surfaces thereof, i.e., the surfaces that may come into contact with a formulation or other substance contained within the pouch, are sterile at the inception or time of formation of the pouch. Thus, the pouch is sealed, empty and sterile from inception. The interior chamber is preferably also substantially particle free and pyrogen free. In addition, if desired, each sealed, empty pouch may be subjected to an additional sterilization process, such as by subjecting each sealed, empty pouch to gamma radiation, ebeam radiation, or by needle injecting a fluid sterilant into the interior of the pouch through its elastic septum. However, as may be recognized by those of ordinary skill in the pertinent art based on the teachings herein, any of numerous other sterilization processes that are currently known, or that later become known, may be employed.

The pouch 22 includes a label-receiving marginal edge portion 124 located on a bottom edge of the pouch. A sealed edge portion 126 defines the left edge of the pouch, a sealed edge portion 128 defines the right edge of the pouch, a sealed portion 130 defines the base of the interior chamber 120 and a fluid-tight barrier between the interior chamber and the label-receiving portion 124, and a sealed edge portion 132 defines the bottom edge of the pouch and the closure to the label-receiving portion 124.

An electronic identifier 79, a label 134 and a dosimeter 136 may be inserted into the label-receiving marginal edge portion 124 prior to sealing one of the edge portions thereof. Then, the label-receiving marginal edge portion 124 is flattened, if necessary, in order to bring the opposing sides of the open outer edge into contact with each other, and the opposing sides are sealed to each other, such as by heat, ultrasonic sealing, or any other desired method, to thereby enclose and retain the respective components within the label-receiving marginal edge portion 124.

In the illustrated embodiment, and as indicated above, the electronic identifier 79 is an RFID tag that provides a unique identifier for the respective pouch 22 and is readable by the radio frequency or RFID transceivers 78 (FIG. 1). As a result, the respective pouch 22 can be traced or tracked at every stage, or at select stages of processing/manufacturing, in order to ensure compliance with quality controls and other manufacturing and/or regulatory procedures and/or guidelines, and if desired, the data of such tracing/monitoring can be recorded by the PLC 80 for future reference. The radiation dosimeter 136 measures the exposure of the dosimeter and thus of the pouch to radiation and undergoes a color change, such as from yellow to red, when the dosimeter is exposed to a sufficient level of radiation, such as gamma or ebeam radiation. Thus, if desired to subject the empty pouch to radiation sterilization as an added measure to ensure sterility, such as gamma or ebeam radiation, the dosimeter 136 will undergo a color change, and the color change will indicate that the pouch has been subjected to a sufficient amount or dose of radiation to ensure that the pouch is sterile and will visibly indicate such exposure and confirmed sterility on the pouch. Thus, the dosimeter provides a visible back-up to the RFID tag 79, which can be used to track and confirm that the pouch has been sterilized, e.g., processed by or in sterilization equipment, if desired to so process the pouch.

As may be recognized by those of ordinary skill in the pertinent art based on the teachings herein, the labels may include any of numerous different features that are currently known, or that later become known, including different features for identifying the pouch, such as a bar code, computer chip, or other optical or electronic device for identifying, tracing and/or monitoring the pouch. Alternatively, the label may include only an electronic identifier, such as an RFID tag. In addition, the label-receiving marginal edge portion 124 need not include a label at all, but rather may receive an electronic identifier, such as an RFID tag, dosimeter and/or other device thereon without a label, or may receive one or more such devices separate from a label. Accordingly, the label-receiving marginal edge portion may serve any of numerous different purposes, and/or may receive any of numerous different devices, that are currently known, or that later become known. Still further, the label-receiving marginal edge portion may be located on any marginal edge or other portion of the pouch, and need not extend along the entire respective edge portion.

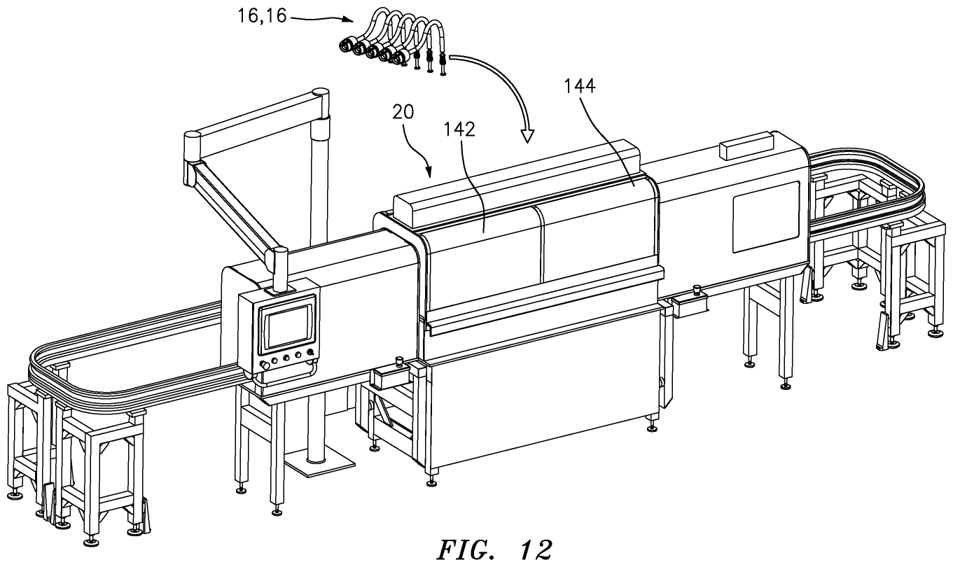

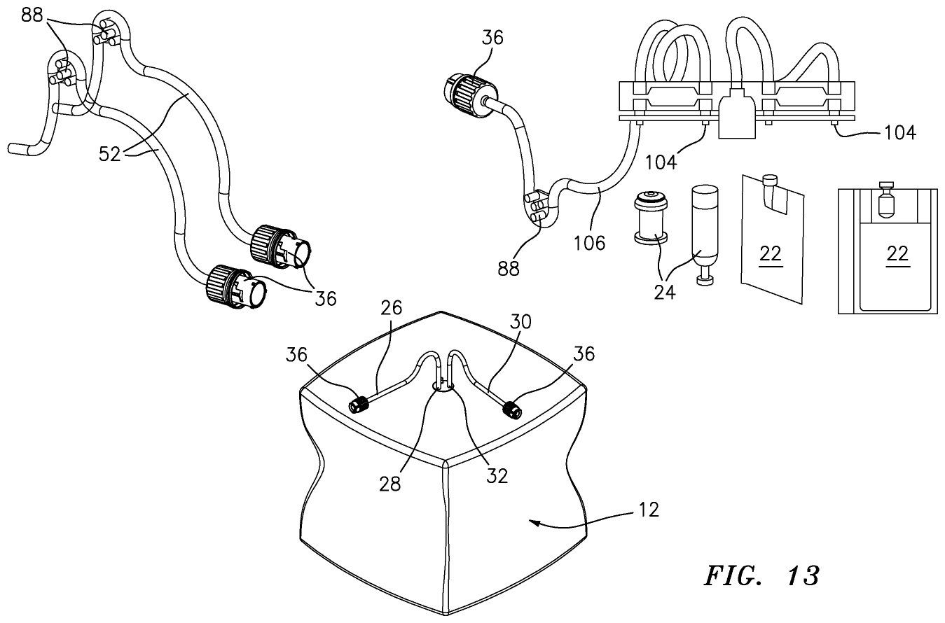

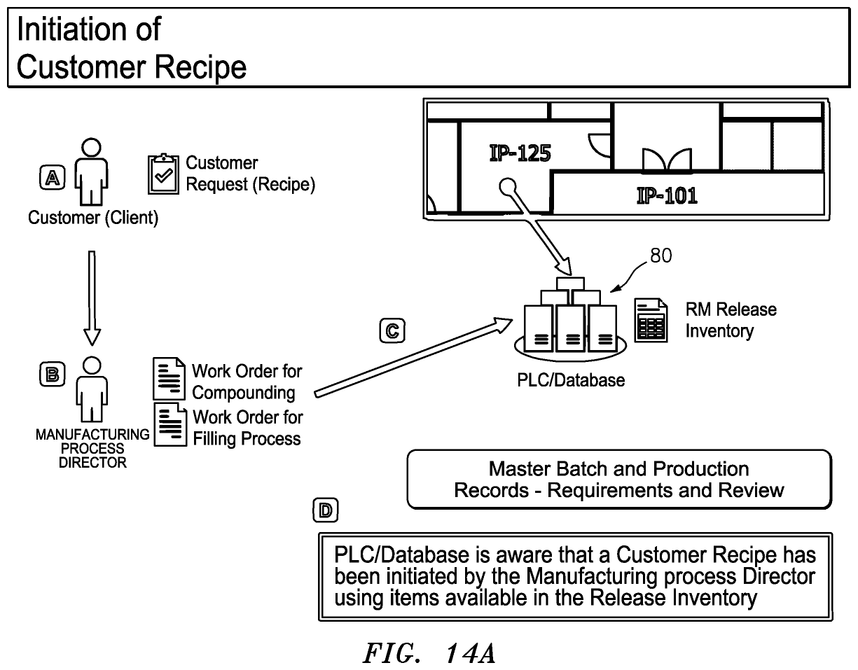

The interior chamber 120 of the pouch is sterile or aseptic filled with a substance by closed sterile transfer in the closed sterile transfer filling assembly 20. The fitment 122 includes dual ports 138 laterally spaced relative to each other and extending outwardly from the marginal end portion 116. Each port 138 includes over-molded therein, and sealed thereto, a respective elastic septum 110, at least one of which may be a needle penetrable and resealable septum. In the illustrated embodiment, each pouch 22 can be needle filled through the left-hand port 138, and as shown in FIG. 11, the penetrated septum 110 can be re-sealed with a liquid sealant 140. In the illustrated embodiment, the liquid sealant 140 is a hot-melt adhesive sealant. As shown in FIG. 12, the closed sterile transfer filling assembly 20 includes a filling station 142 and a sealing station 144 located downstream of the filling station. The filling station 142 includes the closed sterile transfer filling devices 104, and the sealing station includes sealing applicators (not shown) that dispense the hot melt sealant onto the penetrated elastic septa 110 in order to permanently seal the resulting penetration aperture in each septum. As shown in FIG. 11, the hot-melt adhesive sealant 140 overlies the respective septum 110 and is adhesively bonded to the respective port 138 to thereby form a fluid-tight seal between the penetrated septum and the ambient atmosphere. The hot-melt sealant 140 adhesively bonds to the annular wall of the respective port 138 to form an annular, fluid-tight seal between the sealant and the port. Prior to dispensing the hot-melt sealant 140 onto the septum 110, the sealant is heated to a sufficiently high temperature to melt the sealant and have a bactericidal effect on surfaces contacted by the melted sealant. As the liquid sealant 140 flows onto each penetrated septum 110, the temperature of the liquid sealant is sufficiently high to sterilize the contacted surfaces, and thus sterilize the interfaces between the sealant 140 and both the septum 110 and contiguous support surfaces of the respective port 138. Accordingly, the interior of the seal 140 is sterile at the time of formation, and because the sterile interior is sealed with respect to ambient atmosphere, its sterility is maintained throughout the shelf-life and usage of the pouch. As a result, organism growth between the septum 110 and seal 140 may be prevented. The liquid sealant 140 cures at room temperature, and when cured, it forms a solid, rigid, substantially inflexible closure overlying the penetrated septum. In the illustrated embodiment, the hot-melt sealant 140 is a polyolefin (or polyolefin blend), and the septum support of the port 138 is a polypropylene (or polypropylene blend) in order to allow the sealant to firmly bond to the septum support and form a fluid-tight seal therebetween. Thus, in the illustrated embodiment, the hot-melt sealant 140 and septum support of the port 138 are formed of materials with sufficiently common monomers to allow bonding therebetween, and the septum 110 and septum support of the port 138 are also formed of materials with sufficiently common monomers to allow bonding therebetween. Also in the illustrated embodiment, the septum 110 may be formed of a thermoplastic elastomer or a silicone that is bondable to the polypropylene septum support of the port 138. One hot-melt adhesive sealant that may be used is sold by the Minnesota, Mining and Manufacturing Company (“3M”) as 3M Scotch-Weld Hot Melt Adhesive, product no. 3792. As may be recognized by those of ordinary skill in the pertinent art based on the teachings herein, the disclosed materials are only exemplary, and any of numerous other materials that are currently known, or later become known, may be employed.

The sealing station 144 (FIG. 12) may include a temperature sensor (not shown) connected by a wire or wirelessly to the PLC 80. Upon or within a set time following application of the hot-melt sealant 140 to each septum 110, the temperature sensor senses the temperature of the hot-melt sealant and transmits same to the PLC 80. The controller 80 compares the sensed temperature to an acceptable range, or otherwise determines whether the applied sealant temperature is sufficiently high to have a bactericidal effect on the surfaces of the septum. In addition, the temperature sensor may measure the temperature profile across the surface of the septum, to assess whether the hot melt sealant fully covers the septum. For example, if a portion of the scanned temperature profile is below a lower temperature threshold, this may indicate a bare or thin spot in the overlying sealant, and thus, based on the read of the respective electronic identifier 79, the PLC 80 can flag the respective pouch for rejection or further inspection. Alternatively, or in addition to the temperature sensor, a computer vision camera may be used to visually monitor the application of the hot-melt sealant, and flag any seals that do not meet or substantially conform to a required visual seal profile. The PLC 80 can match the reading of the computer vision camera to the respective pouch based on its electronic identifier 79, and the respective pouch can be flagged for rejection or further inspection if the image of the seal does not meet or substantially conform to a required visual seal profile.

As shown in FIG. 11, the septum 110 of the right-hand port 138 is exposed in the illustrated condition of the pouch to allow connection thereto of a sterile connector, such as one of the sterile connectors 36, 36, in order to withdraw substance from the pouch therethrough. The right-hand septum 110 may be covered with a tamper-resistant closure (not shown), such as an adhesive-backed foil or other covering, that requires removal of the closure in order to access the septum. In other embodiments, one or more of the ports 138 may be a valve for filling substance into and/or withdrawing substance from the pouch 22. Examples of valves that may be utilized are disclosed in U.S. patent application Ser. No. 14/990,778, filed Jan. 7, 2016, entitled “Pouch with Sealed Fitment and Method,” which claims the benefit of similarly-titled U.S. Provisional Patent Application No. 62/100,725, filed Jan. 7, 2015, which are hereby incorporated by reference in their entireties as part of the present disclosure.

The pouches and other primary containers or devices of the present disclosure, and the methods of making and filling such primary containers or devices, may be the same as or similar to the containers or devices and methods disclosed in the following co-pending patent applications, which are hereby incorporated by reference in their entireties as part of the present disclosure: U.S. patent application Ser. No. 14/990,778, filed Jan. 7, 2016, entitled “Pouch With Sealed Fitment and Method,” which claims the benefit of similarly-titled U.S. Provisional Patent Application No. 62/100,725, filed Jan. 7, 2015; and U.S. patent application Ser. No. 15/410,740, filed Jan. 19, 2017, entitled “Pouch With Fitment and Method of Making Same,” which claims the benefit of U.S. Provisional Patent Application No. 62/280,700, filed 19 Jan. 2016, entitled “Pouch with Heat-Sealed External Fitment,” U.S. Provisional Patent Application No. 62/295,139, filed Feb. 14, 2016, entitled “Pouch with Over-Molded Fitment and Method of Making Same,” U.S. Provisional Patent Application No. 62/298,214, filed Feb. 22, 2016, entitled “Pouch with Over-Molded Fitment and Method of Making Same,” U.S. Provisional Patent Application No. 62/323,561, filed Apr. 15, 2016, entitled “Pouch with Over-Molded Fitment and Method of Making Same,” and U.S. Provisional Patent Application No. 62/448,315, filed Jan. 19, 2017, entitled “Pouch With Fitment and Method of Making Same.”

As shown in FIG. 1, the apparatus 10 includes a formulating enclosure 146, a filling enclosure 148, and a finished product enclosure 150. The formulation and filling enclosures 146 and 148, respectively, may each include an over-pressure of microfiltered air or other gas, such as by employing one or more HEPA filters (not shown). The formulation enclosure 146 includes one or more access doors or ports 152, the filling enclosure 148 includes one or more access doors or ports 154, and the finished product enclosure 150 includes one or more access doors or ports 156. Each access door or port 152, 154, 156 includes an associated electronic reader or scanner 78 that reads the electronic identifier 79 on each primary device and process device that passes through the respective door or port, and transmits the readings to the PLC 80 to, in turn, trace, monitor and record the passage of such devices into and out of the respective enclosure. Any other articles or persons that enter or exit the formulation enclosure 146, filling enclosure 148, or finished product enclosure 150 may similarly bear a respective electronic identifier 79 to trace, monitor and record the passage of such articles or persons into and out of the respective enclosure. In addition, each access door or port 152, 154, 156 may include an electronic lock controlled by the PLC 80 where the PLC only opens the lock, and thus opens or allows to be opened the respective access door or port, when the correct devices, articles and/or persons are present at the door, and/or when such devices, articles and/or persons are in the requisite condition for passage through the door. For example, if the empty primary containers or devices or process containers or devices must be sterilized prior to entry into the formulation enclosure 146 and/or filling enclosure 148, the PLC 80 can determine by reading the respective electronic identifier 79 whether the container or device has been subjected to the requisite sterilization process. If not, the PLC 80 may be programmed to not open the respective access door 152 or 154, or alternatively, to generate an alarm or otherwise flag the respective device or container for rejection or further inspection. As may be recognized by those of ordinary skill in the pertinent art based on the teachings herein, the formulating enclosure 146, filling enclosure 148, and finished product enclosure 150, each may take the form of any of numerous different types of enclosures that are currently known or that later become known. In one embodiment, each such enclosure is defined by a room with doors and an overpressure of microfiltered air or other gas such then when the doors are opened, the air and/or other gas flows through the door in the direction out of, rather than into the room. In other embodiments, each such enclosure may be simply a cage, such as a wire fencing with apertures in the fencing, or a partially enclosed area, or may be simply an area where the respective equipment is located without any enclosure surrounding or otherwise enclosing the respective equipment.

When manufacturing a respective product/formulation, the PLC 80 includes the specification or “recipe” for the respective product/formulation. The specification identifies the required formulation ingredients, relevant proportions of such ingredients, primary containers or devices, process containers or devices, the connections that need to be made by each required inlet closed sterile transfer assembly, the connections required to be made by each required outlet closed sterile transfer assembly, the required flow rates for the respective formulation components/ingredients, and any other required formulation or filling processing parameters. The PLC 80 may be programmed to prevent the formulation or filling process from proceeding, or otherwise to generate an alarm, if any requisite conditions of the specification are not met. For example, if an inlet closed sterile transfer assembly is connected to the wrong outlet closed sterile transfer assembly, if a sterile connector assembly is received within the wrong cradle or is not fully connected or properly received in its respective cradle, if a pump is not working, or if a pump is operating at the wrong speed such that the flow rate of the respective formulation ingredient or formulation is not in accordance with the specification, the PLC 80 generates an alarm, identifies the aspect of the equipment or process not in accordance with the specification, and flags the defect or error for correction, preferably before the respective process can further proceed. As indicated above, the PLC 80 can be connected to each pump 88 in order to control the operation of the pump. The PLC 80 controls the operation of each pump such that the respective formulation ingredient(s) or formulation is processed in accordance with the specification. If the operation of any pump or sensor signal transmitted to the PLC is not in accordance with the specification, the PLC generates an alarm or otherwise flags the respective primary or process container or device for rejection, inspection or other corrective action.

As indicated in FIG. 1, the intermediate or surge tank 14 may be located in the filling enclosure 148 and connected through a respective access door or port 154 to the formulation tank 12 in the formulation enclosure 146. Alternatively, the surge tank 14 may be filled from the formulation tank 12 by closed sterile transfer in the formulation enclosure 146, disconnected from the formulation tank 12, and transported out of the formulation enclosure 146 through its access door 152 and, in turn, moved into the filling enclosure 148 through its access door 154. As with the other primary and process containers or devices, the PLC 80 traces, monitors and records the processing of the surge tank 14 based on the signals transmitted by the electronic scanners 78 as they detect the respective electronic identifier 79 upon passage of the surge tank through the respective doors/ports.

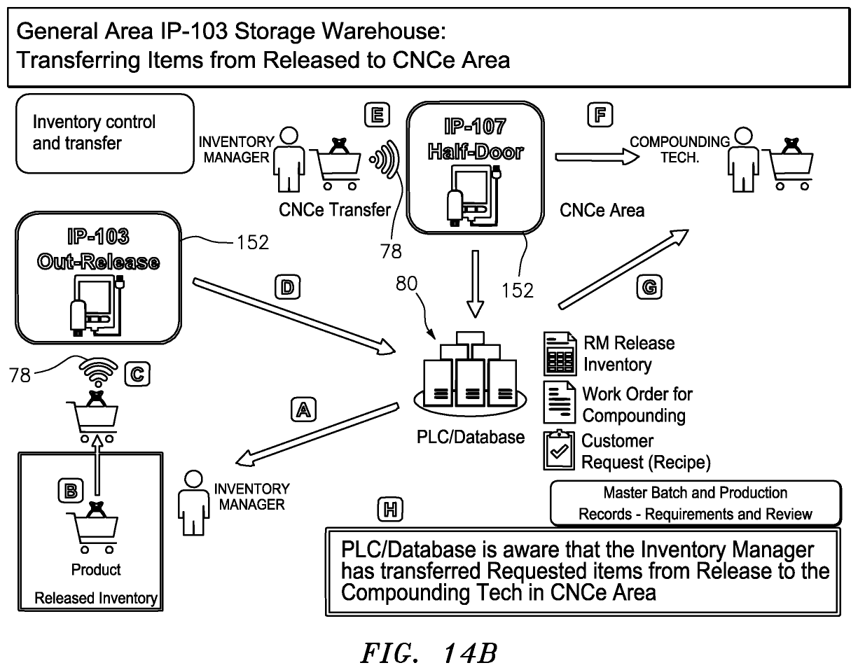

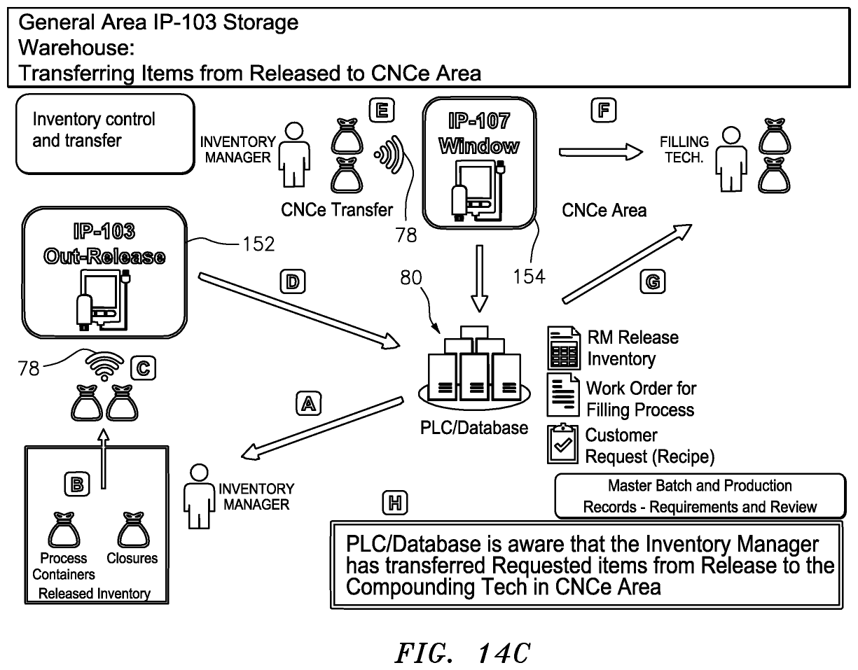

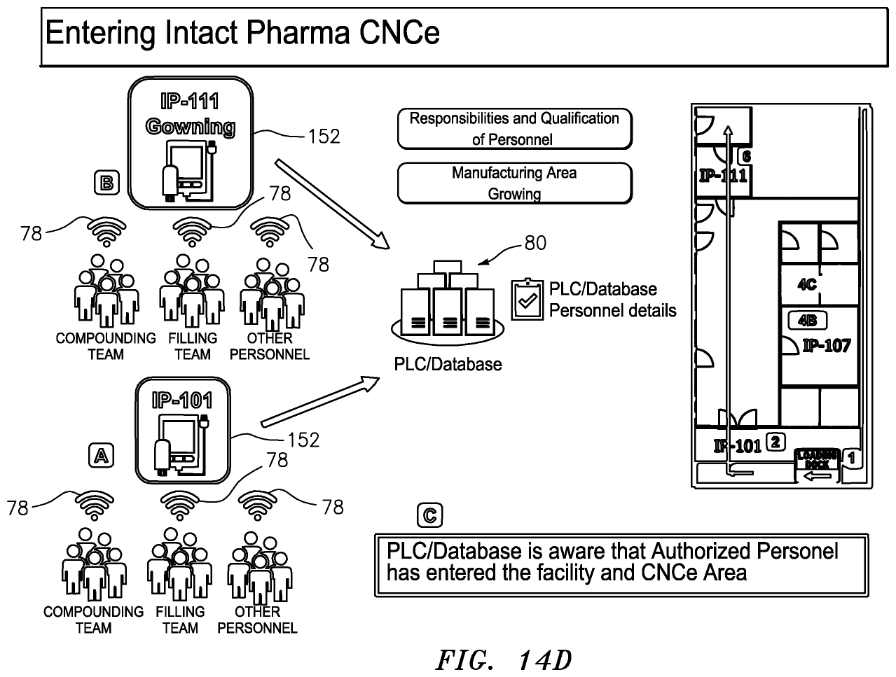

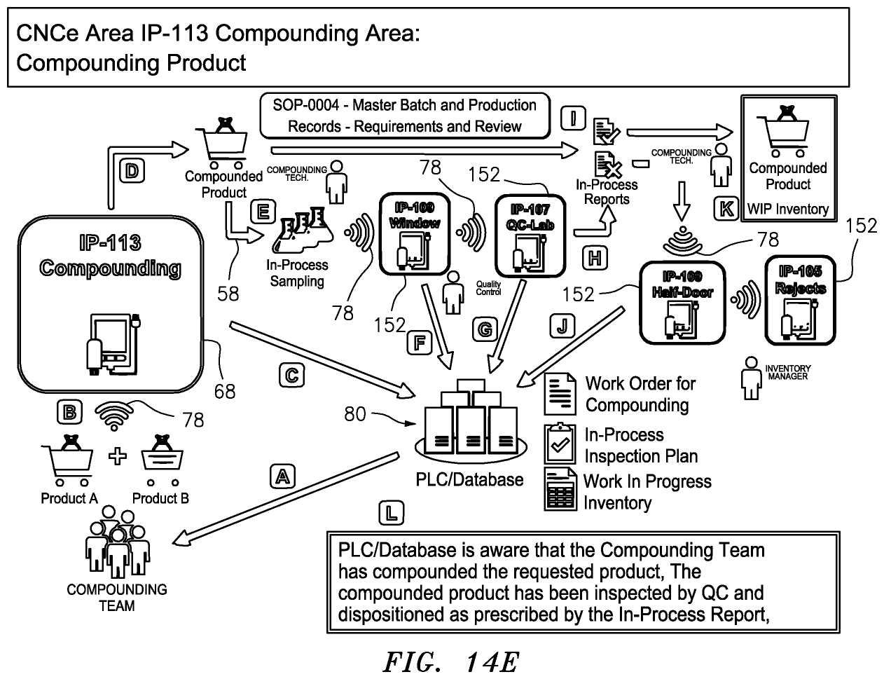

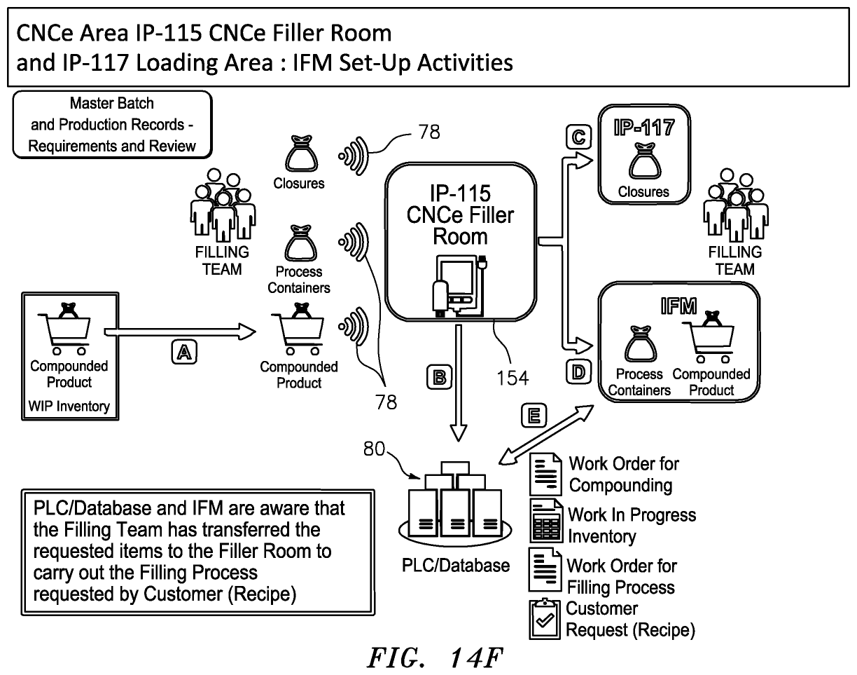

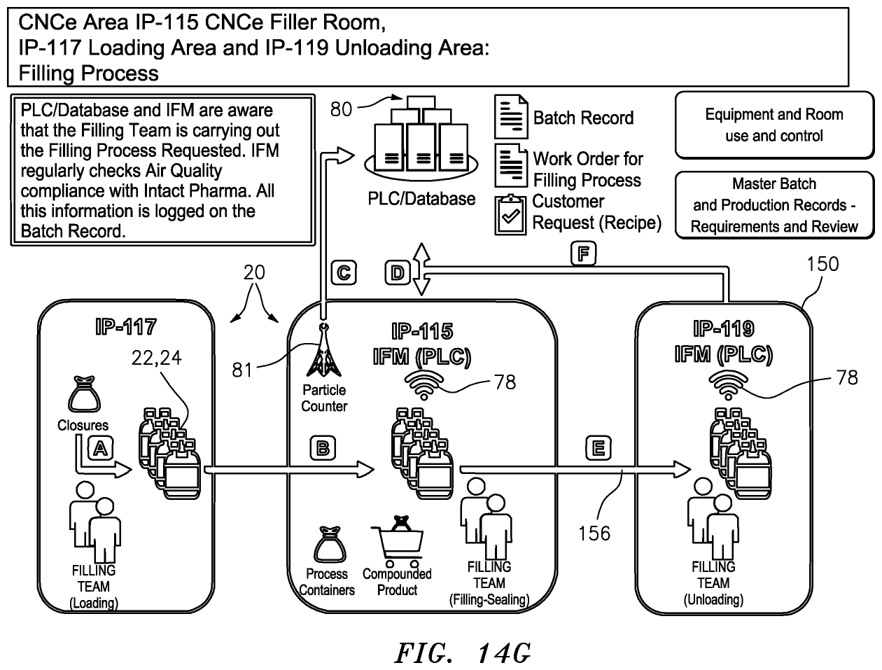

Turning to FIG. 14A, the production of a recipe, such as a customer recipe, is initiated by identifying on the PLC 80 the respective customer request (or recipe or specification). As indicated, the PLC 80 includes or otherwise accesses a database containing the customer request, the work order for compounding the respective recipe in the formulation enclosure 146, the work order for filling the respective recipe in the filling enclosure 148, and the inventory required for the compounding and filling work orders, i.e., the “release inventory.” As indicated in FIG. 14B, the PLC 80 traces the release of the required inventory from the release inventory for delivery to the formulation enclosure 146. The PLC 80 transmits to the compounding technician the customer request, work order for compounding, and required release inventory. When the compounding technician pulls the release inventory, the transceiver 78 at the respective “out release” door 152 reads the electronic identifiers 79 on each item of inventory and transmits same to the PLC 80 to trace the actual inventory released by the inventory manager and allow the PLC to confirm that the released inventory matches the required release inventory for the respective request. If the release inventory is not correct, the PLC 80 notifies the inventory manager and prevents the formulation processing from further proceeding until the release inventory is correct. Then, when the release inventory is introduced through the access door 152 and into the compounding enclosure 146, the transceiver 78 at the respective access door 152 reads the electronic identifier 79 on each item of release inventory introduced into the compounding enclosure 146, and transmits same to the PLC 80 to trace the actual inventory introduced into the compounding enclosure and allow the PLC to confirm that the released inventory matches the required release inventory for the respective request. If the release inventory introduced into the compounding enclosure 146 is not correct, the PLC 80 notifies the compounding technician and prevents the formulation processing from further proceeding until the release inventory is correct.

As shown in FIG. 14C, the PLC 80 also traces the release of the required inventory from the release inventory for delivery to the filling enclosure 148. When the inventory manager releases the inventory for the filling process, the transceiver 78 at the respective “out release” door 152 reads the electronic identifiers 79 on each item of inventory and transmits same to the PLC 80 to trace the actual inventory released by the inventory manager and allow the PLC to confirm that the released inventory matches the required release inventory for the respective request. If the release inventory is not correct, the PLC 80 notifies the inventory manager and prevents the filling processing from further proceeding until the release inventory is correct. Then, when the release inventory is introduced through the access door 154 and into the filling enclosure 148, the transceiver 78 at the respective access door 154 reads the electronic identifier 79 on each item of release inventory introduced into the filling enclosure 148, and transmits same to the PLC 80 to trace the actual inventory introduced into the filling enclosure and allow the PLC to confirm that the released inventory matches the required release inventory for the respective request. If the release inventory introduced into the filling enclosure 148 is not correct, the PLC 80 notifies the filling technician and prevents the fill processing from further proceeding until the release inventory is correct.

As shown in FIG. 14D, the PLC 80 traces the personnel entering the formulation and filling enclosures to confirm that only authorized personnel enter, and are properly gowned, if so required, before doing so. The entry door 152 to the gowning area includes a respective transceiver 78 that transmits to the PLC 80 the identities of the personnel entering the gowning area or otherwise entering the formulation/filling enclosures. Each authorized person wears a respective electronic identifier 79 or is required to otherwise confirm identity through an electronic finger print reader, optical reader, or other electronic personnel identification device at the entry door. As may be recognized by those of ordinary skill in the pertinent art based on the teachings herein, the devices and/or methods for identifying authorized personnel may take the form of any of numerous different devices or methods that are currently known, or that later become known. The exit door 152 of the gowning area similarly includes a transceiver 78 for transmitting to the PLC 80 confirmation that each authorized person, and only authorized personnel, exits the gowning area to the formulation and/or filling enclosures.

As shown in FIG. 14E, the PLC 80 monitors whether the compounding team has compounded the requested product, and whether the compounded product is dispositioned as prescribed in the respective specification. The PLC 80 transmits to the compounding team in the compounding enclosure 146 the work order for compounding, the in-process inspection plan, and the work-in-progress inventory. The compounding cradle(s) 68 in the compounding enclosure transmit to the PLC 80 respective compounding information, including that the respective sterile connector(s) 36, 36 are received therein and the flow rates of the formulation components therethrough. The PLC 80 checks the data transmitted by each cradle 68 against the specification of the respective work order. If the sterile connectors are not matched correctly, or otherwise not connected correctly, the respective cradle(s) 68 transmit signals indicative thereof to the PLC 80. If, on the other hand, the flow rate signals are not in accordance with the specification, the PLC 80 can control the respective pump 88 to correct the respective flow rate. Otherwise, if any transmitted data is not in accordance with the specification, the PLC 80 can transmit an alarm to the compounding team to take corrective action or otherwise flag the respective product for further inspection or rejection. Any in-process samples taken by the sampling device 58 are traced by a respective electronic identifier 79 on the sampling device or sample vial or other container. A transceiver 78 mounted at the respective outlet door 152 and another transceiver 78 mounted at the inlet door 152 of a quality control lab allows the PLC 80 to trace the passage of each sample from the compounding enclosure to the quality control lab. The results of the testing at the quality control lab are transmitted to the PLC 80 as well. If the test results are in accordance with the respective specification, the PLC allows the compounding to proceed or allows for acceptance of the compounded formulation. If, on the other hand, the test results do not comply with the respective specification, the PLC 80 transmits an alarm or other message to the compounding team to take corrective action, and otherwise, depending on the test results, prevent further compounding or flag the respective formulation for further inspection or rejection. If the formulation passes the quality control testing, it is passed through an outlet door 152 of the compounding enclosure and either delivered to a work in progress inventory or to the filling enclosure 148. The PLC 80 traces the movement of the formulation as it passes through the outlet door based on the signal transmitted by the respective transceiver 78. If, on the other hand, the formulation is rejected, such as for failure to pass the quality control testing, it is transported through an access door 152 to a “rejects” enclosure and such movement is traced by the PLC 80 based on the signals transmitted by the respective access door transceiver 78.

Turning to FIG. 14F, in order to conduct a filling operation, the PLC 80 transmits to the filling team in the filling enclosure 148 the work order for compounding, the work in progress inventory, the work order for the filling process, and the customer request or recipe. The electronic identifiers 79 on the compounded product, the process containers or devices, the primary container or devices (or “closures”), and each authorized person on the filling team, allows the transceiver(s) 78 located at the access door 154 to the filling enclosure 148 to transmit the identification information to the PLC 80 to, in turn, trace their passage into the filling enclosure 148. If any such item or person is not the correct item or person, or if any such item or person has not undergone the correct pre-processing or otherwise is not in compliance with the respective specification, the PLC 80 generates an alarm or other message to the filling team to take corrective action before allowing the fill processing to further proceed.

Turning to FIG. 14G, the filling team loads the sealed, empty, sterile primary containers or devices 22, 24 to be filled into the closed sterile transfer filling assembly 20. A particle counter 81 may be mounted within the filling enclosure 148 or otherwise on the closed sterile transfer filling assembly 20 to monitor the air quality, such as by counting the particles in the air, and transmits the data to the PLC 80. If the air quality is not in compliance with the specification, the PLC 80 can generate an alarm to take corrective action and/or terminate the fill processing. The transceiver 78 mounted on or adjacent to the closed sterile transfer filling assembly 20 traces, monitors and records the primary containers or devices as they travel through each stage of fill processing, including any pre-penetrable surface sterilization, closed sterile transfer filling, and resealing of the resulting piercing member/needle penetration holes in the elastic septa. If any such container or device does not pass through all required fill processing steps or stages, or if the closed sterile transfer filling assembly transmits data to the PLC 80 indicating that the respective primary container or device was not filled or otherwise processed in accordance with the specification (e.g., if the respective sensor indicates that a resulting piercing member/needle hole may not be fully or otherwise properly resealed), the PLC 80 flags the respective primary container or device for further inspection and/or rejection. For example, each needle penetrable port 138 including the penetrable septum 110 of each primary device 22 is received within a fixture of the filling station 142 (not shown) that includes a marking, such as a laser etched line, that is spaced a predetermined distance from each port or septum when properly received and positioned therein for needle penetration of the septum. A sensing camera (not shown) is connected to the PLC 80 and measures the distance between the marking and the edge or other surface of the port or septum. If the measured distance is greater than a maximum predetermined distance, the PLC 80 generates an alarm indicating that the respective port/septum is not properly seated in the filling fixture to, in turn, allow a fill operator to take corrective action and/or it flags the respective primary device for inspection or rejection. The filling machine 20 includes a transceiver 78 at each respective station, such as at a surface decontamination station (e.g., by UV irradiation) where the penetrable surface of each septum 110 is decontaminated prior to needle penetration, at the needle penetration and filling station, and at the resealing station. The PLC 80 records and generates a time stamp upon or at entry of each primary device into the respective station. If the time period between time stamps from one station to the next is outside of the specification (e.g., if the time period is too long or too short), the PLC 80 flags the respective primary device(s) and generates an alarm to further inspect and/or reject the flagged device(s). The filling machine 20 may include additional sensors, such as sensors for measuring whether the closure or shutter on each filling needle is closed when the needle is in the withdrawn or “up” position, a sensor for measuring the piercing force of each filling needle as it penetrates each septum 110, and other sensors that measure the temperature of the liquid sealant. The PLC 80 compares the sensor readings to required readings for each such sensor, and if any readings are outside the respective specification, the PLC 80 flags the respective devices that were undergoing processing at the respective station at the time of the faulty or unacceptable readings, and otherwise generates an alarm to the operator to inspect and take corrective action. Upon passage of the filled products through the access door 156 to the finished product enclosure 150, the respective transceiver 78 scans or otherwise reads the electronic identifiers on the filled primary containers or devices and thereby allows the PLC 80 to trace, monitor and record the passage of such devices into the unloading area.

As may be recognized by those of ordinary skill in the pertinent art based on the teachings herein, numerous changes, modifications and improvements may be made to the above-described and other embodiments without departing from the scope of the invention as defined in the appended claims. For example, the primary and process devices, the compounding/formulation components, the filling machines, the electronic identifiers, the electronic readers, the PLC and other above-described components or devices may take the form of any of numerous different components or devices that are currently known, or that later become known. In addition, the PLC or other controller(s) may be programmed to operate in accordance with any of numerous different methods, that are currently known, or that later become known. Accordingly, this detailed description of embodiments is to be taken in an illustrative, as opposed to a limiting sense.

What is claimed is:

1. A method comprising:
(i) reading identification information defined by electronic identifiers on one or more primary devices or process devices;
(ii) transmitting the read identification information to a controller, comparing the read identification information to required identification information for a respective specification, and transmitting a signal to further proceed or not based on the comparison; and
(iii) if a signal to further proceed is transmitted, transferring by closed sterile transfer one or more substances from said one or more primary devices to said one or more process devices or from said one or more process devices to said one or more primary devices.

2. A method as defined in claim 1, wherein the identification information includes first information identifying the respective device and distinguishing the device from other devices.

3. A method as defined in claim 2, wherein the identification information further includes second information on the condition or processing status of the respective device.

4. A method as defined in claim 3, wherein the second information includes whether the respective device was subjected to sterilization in a sealed, empty state.

5. A method as defined in any one of claims 1 to 4, wherein the closed sterile transfer is without exposure of the transferred substance to the ambient atmosphere, and the transferred substance is sealed with respect to the ambient atmosphere.

6. A method as defined in claim 5, wherein the primary device includes formulation component containers and formulation containers, and step (iii) includes transferring by closed sterile transfer a plurality of formulation components from respective component containers to a formulation container and combining the formulation components into a formulation in the formulation container.

7. A method as defined in claim 6, wherein the process devices include sterile connector assemblies.

8. A method as defined in claim 7, wherein each sterile connector assembly includes a first connector and a second connector, the first and second connectors are connectable to each other and configured to transfer substance through the sterile connector assembly by closed sterile transfer.

9. A method as defined in claim 8, wherein each sterile connector assembly includes at least one of said electronic identifiers, and is receivable within a respective connector support, and the connector support includes a reader configured to read the identification information of said electronic identifier of the sterile connector assembly, and the method further comprises transmitting a signal to a controller indicative of the identification information of the respective sterile connector, comparing the identification information to required identification information for the respective support, and further proceeding or not based on the comparison.

10. A method as defined in claim 9, measuring at the connector support a flow rate of a formulation or one or more formulation components flowing through the respective sterile connector, transmitting to the controller a signal indicative of the measured flow rate, and comparing the measured flow rate to a required flow rate for the respective formulation or one or more formulation components.

11. A method as defined in claim 3, wherein the identifiers are on plural component containers and each component container contains one or more respective formulation components sealed with respect to ambient atmosphere in the component container.

12. A method as defined in any one of claims 6 to 11, further comprising reading identification information electronic identifiers on plural component containers, transmitting read electronic identification data to a controller, comparing via the controller the read electronic identification data to required identification data for a respective formulation, and transmitting via the controller a signal to proceed with step (iii) if the read electronic identification data substantially matches the required identification data for a respective formulation.

13. A method as defined in any one of claims 6 to 11, further comprising reading identification information of electronic identifiers on plural component containers, plural sterile connectors, and at least one formulation container, transmitting read electronic identification data to a controller, comparing via the controller the read electronic identification data to required identification data for a respective formulation, and transmitting via the controller a signal to proceed with step (iii) if the read electronic identification data substantially matches the required identification data for a respective formulation.

14. A method as defined in any one claims 1 to 13, further comprising transferring by closed sterile transfer a formulation from a formulation container to a dispensing container, wherein the closed sterile transfer is without exposure of the formulation to the ambient atmosphere, and the formulation is sealed with respect to ambient atmosphere in the dispensing container.

15. A method as defined in claim 14, further comprising storing multiple doses of the formulation in a storage chamber of the dispensing container, maintaining the formulation in the storage chamber sterile and hermetically sealed with respect to the ambient atmosphere during storage and during dispensing of doses from the storage chamber.

16. A method as defined in claim 14 or 15, wherein the closed sterile transfer is further without exposure of the formulation or formulation components thereof to germs or other contaminants.

17. A method as defined in any one of claims 14 to 16, wherein the closed sterile transfer further includes moving a piercing member and/or an elastic septum relative to the other between a first position where the piercing member is not penetrating the septum and a second position where the piercing member is penetrating the septum, decontaminating the piercing member by physical interaction with the elastic septum during movement between the first position and the second position, and introducing the formulation or formulation components thereof through the piercing member in the second position.

18. A method as defined in any one of claims 14 to 17, further comprising sterile closed transfer filling plural dispensing containers, reading identification information of electronic identifiers of each such container, and tracing each such container through plural steps of the closed transfer filling process by reading the identification information of its electronic identifier at each such step of the process.

19. A method as defined in any one of claims 1 to 18, further comprising (i) sterilizing at least one first formulation component in a first component container; (ii) sterilizing at least one second formulation component in a second component container; (iii) transferring by closed sterile transfer the at least one first formulation component from the first component container to a formulation container; and (iv) transferring by closed sterile transfer the at least one second formulation component from the second component container to said formulation container.

20. A method as defined in claim 19, further comprising closed sterile transferring the at least one first and second formulation components to the formulation container through respective closed sterile transfer connector assemblies, reading identification information of an electronic identifier of each formulation container and of each sterile connector assembly, determining based on the read identification information whether the formulation containers and sterile connectors are correctly connected, and proceeding or not based on the determination.

21. A method as defined in claim 19 or 20, further comprising sterilizing the at least one first formulation component by relatively cold sterilization, and sterilizing the at least one second formulation component by relatively hot sterilization.

22. A method as defined in claim 21, wherein the cold sterilization includes subjecting the at least first formulation component to ebeam irradiation sterilization and/or microfiltration sterilization, and the hot sterilization includes thermal sterilization.

23. A method as defined in any one of claims 20 to 22, wherein the at least one first formulation component and/or the least one second formulation component is sterilized prior to introduction into the respective component container.

24. A method as defined in claim 6, further comprising (i) connecting a respective sterile connector assembly in fluid communication between each respective component container and the formulation container, wherein the closed sterile transfer includes transferring a respective formulation component through each respective sterile connector assembly, and (ii) connecting at least one sterile connector assembly in fluid communication between the formulation container and at least one dispensing container, wherein the closed sterile transfer includes transferring the formulation from the formulation container to the at least one dispensing container through the at least one sterile connector assembly.

25. A method as defined in claim 24, further comprising reading identification information of electronic identifiers of each respective sterile connector and each respective component container, determining based on the read identification information whether each respective sterile connector is correctly connected to each respective component container, and proceeding or not based on said determination.

26. A method as defined in any one of claims 1 to 25, further comprising introducing a plurality of primary devices and process devices into a formulation enclosure, wherein the primary devices are sealed and empty; upon or during passage into the formulation enclosure, reading identification information of electronic identifiers of at least a plurality of such primary devices and/or process devices; determining with the controller if any such device was not sterilized but should have been sterilized based on the read identification information; and generating a signal indicating if any such device was not sterilized.

27. A method as defined in claim 26, wherein the process devices include sterile connector assemblies and the primary devices include formulation component containers, and the method further includes connecting the formulation component containers to a formulation container with the sterile connector assemblies; placing each of a plurality of connected sterile connectors in respective connector supports; reading with a sensor of each connector support identification information of the respective connector in the support; transmitting the read connector identification information to the controller; and comparing the read connector information to required connector information.

28. An apparatus comprising:
a plurality of primary devices or process devices, wherein each device is sealed, empty and includes an electronic identifier defining identification information;
one or more of a formulation enclosure or a filling enclosure, wherein each enclosure includes a door for the passage of one or more of primary devices or process devices into and/or out of the enclosure;
a scanner configured to read the electronic identifiers and identification information defined thereby prior to, during or upon passage through the door, and transmitting the read identification information; and
a controller configured to receive the read identification information from the scanner, compare the read identification information to required identification information for a respective specification, and transmit a signal to further proceed with a process in the enclosure or not based on the comparison.

29. An apparatus as defined in claim 28, wherein the enclosure is a formulation enclosure, the primary devices include plural component containers and at least one formulation container, and the process devices include plural sterile connectors.

30. An apparatus as defined in claim 28, wherein the enclosure is a filling enclosure, the primary devices include plural dispensing devices or containers, and the process devices include filling kits.

31. An apparatus as defined in claim 30, wherein each filling kit includes a conduit, a sterile connector located at one end of the conduit, and a filling head located at another end of the conduit, wherein the sterile connector is configured to transfer substance by sterile closed transfer into the conduit and to the filling head, and the filling head is configured to transfer by closed sterile transfer the substance from the conduit into the dispensing devices or containers.

32. An apparatus as defined in claim 30, wherein each of a plurality of sterile connector assemblies is sealingly connected in fluid communication with a respective flexible tubular conduit, and the apparatus further comprises a peristaltic pump that engages the exterior of the flexible tubular conduit for pumping substance through the tubular conduit.

33. An apparatus as defined in claim 28, further comprising:
a formulation container including a formulation chamber that is sealed with respect to ambient atmosphere, a plurality of inlet ports in fluid communication with the formulation chamber, at least one outlet port in fluid communication with the formulation chamber, and an electronic identifier; and
a plurality of sterile connectors, wherein a plurality of the sterile connectors are each sealingly connected in fluid communication with a respective inlet port; at least one sterile connector is sealingly connected to the outlet port; each sterile connector is normally closed and seals the formulation chamber with respect to ambient atmosphere; each sterile connector is engageable with another sterile connector and connectable in fluid communication therewith to form a sterile connector assembly; at least one of each sterile connector or one or more sterile connectors within the sterile connector assembly includes an electronic identifier; and each sterile connector assembly defines a closed conduit therethrough that is sterile and sealed with respect to ambient atmosphere for preventing exposure of any formulation components or formulation flowing therethrough to the ambient atmosphere to thereby (i) allow closed sterile transfer of formulation components through the sterile connector and into the formulation chamber, or (ii) allow closed sterile transfer of formulation out of the formulation chamber and through the sterile connector.

34. An apparatus as defined in claim 33, wherein each sterile connector includes either a piercing member or an elastic septum, and is engageable with another sterile connector including the other of a piercing member or an elastic septum to form a sterile connector assembly.

35. An apparatus as defined in claim 34, wherein each piercing member and/or elastic septum is movable relative to the other between a first position where the piercing member is not penetrating the septum and a second position where the piercing member is penetrating the septum, the piercing member is decontaminated by physical interaction with the elastic septum during movement between the first position and the second position, and the interior of the piercing member defines the closed sterile conduit for the flow of substance therethrough in the second position.

36. An apparatus as defined in claim 34 or 35, further comprising a plurality of sterile connector assemblies, wherein each sterile connector assembly, or one or more sterile connectors within each sterile connector assembly, includes an electronic identifier, each sterile connector assembly comprises a male connector including a piercing member, and a female connector including an elastic septum, the male and/or female connectors are movable relative to each other between disengaged and engaged positions, during movement between the disengaged and engaged positions, the piercing member penetrates the elastic septum and the elastic septum decontaminates the piercing member by physical interaction therewith, and in the engaged position, the sterile formulation components or formulation are flowable through the sterile connector assembly and sealed with respect to the ambient atmosphere.

37. An apparatus as defined in any one of claims 34 to 36, wherein each piercing member includes an outflow aperture and a closure movable between a closed position covering the outflow aperture, and an open position exposing the outflow aperture, wherein the closure defines a locked condition and an unlocked condition, the closure is in the locked condition prior to and during penetration of the elastic septum, and is in the unlocked condition after the outflow aperture penetrates the septum and allows movement of at least one of the closure or piercing member relative to the other to expose the outflow aperture and allow the flow of a formulation component or formulation therethrough.

38. An apparatus as defined in claim 28, wherein the enclosure is a closed transfer filling enclosure, the scanner is configured to read electronic identifiers and identification information defined thereby of plural dispensing containers, and the controller is configured to trace each such dispensing container through plural steps of a closed transfer filling process by reading said electronic identifiers and identification information defined thereby at each such step of the process.

39. An apparatus as defined in claim 28, wherein the enclosure is a closed transfer formulation enclosure, the scanner is configured to read electronic identifiers and identification information defined thereby of plural process containers and devices, and the controller is configured to trace each such container and device through plural steps of a closed transfer formulation process by reading said electronic identifiers and identification information defined thereby at each such step of the process.

40. An apparatus as defined in any one claims 28 to 39, wherein the scanner is configured to read the electronic identifiers and identification information defined thereby of a plurality of primary and process containers or devices at each of a plurality of processing steps during one or more of a sterilization process, a formulation process, and a filling process, and the controller is configured to trace and record the processing of each such primary and process container or device at each such step.

41. An apparatus as defined in claim 40, wherein the controller is further configured to compare the information transmitted by the scanner to required processing steps for each primary or process container or device during one or more of the sterilization, formulation, and filling processes, and to flag or reject a respective primary or process container or device where the transmitted information does not match a required processing step.

42. An apparatus as defined in claim 41, further comprising one or more databases associated with the controller and configured to record the processing of each primary or process container or device at each required processing step during one or more of the sterilization, formulation and filling processes.

43. An apparatus as defined in any one of claims 33 to 37, further comprising a sampling valve connected in fluid communication with the formulation chamber, wherein the sampling valve includes an electronic identifier, is normally closed and seals the interior of the formulation container with respect to the ambient atmosphere, and is movable between the normally closed position and an open position to sample the formulation components and/or formulation in the formulation chamber.

44. An apparatus as defined in claim 43, wherein the sampling valve includes a valve member, a valve seat, and a spring that normally biases the valve member into engagement with the valve seat to close the valve, wherein the valve member is engageable with a sampling device to move the valve member away from the valve seat against the bias of the spring and open the valve, draw a sample of the formulation components and/or formulation through the open valve, and is disengageable from the sampling device to allow the spring to bias the valve member into engagement with the valve seat, close the valve and seal the formulation chamber with respect to the ambient atmosphere.

45. An apparatus as defined in claim 44, wherein the spring is an elastic spring defining a least one aperture in fluid communication with the formulation chamber for allowing formulation components and/or formulation to flow from the formulation chamber through the aperture and open valve and into the sampling device.

46. An apparatus as defined in claim 43 or 44, further comprising a sampling device including a piston or plunger that is movable to draw a sample of formulation components and/or formulation through the sampling valve and into the sampling device.

47. An apparatus as defined in any one of claims 33 to 37 and 43 to 46, further comprising an intermediate formulation container including an intermediate formulation chamber that is sealed with respect to ambient atmosphere, an inlet port in communication with the intermediate formulation chamber, and an outlet port in fluid communication with the intermediate formulation chamber; a sterile connector sealingly connected in fluid communication with the inlet port; and another sterile connector sealingly connected to the outlet port, wherein each sterile connector is normally closed and seals the intermediate formulation chamber with respect to ambient atmosphere, each sterile connector is engageable with another sterile connector and connectable in fluid communication therewith to form a sterile connector assembly, and (i) allow closed sterile transfer of formulation through the sterile connector and into the intermediate formulation chamber, or (ii) allow closed sterile transfer of formulation out of the intermediate formulation chamber and through the sterile connector, wherein the closed sterile transfer is without exposure of the formulation to the ambient atmosphere, and the formulation is sealed with respect to ambient atmosphere; and one or more electronic identifiers.

48. An apparatus as defined in claim 47, further comprising a closed transfer filling assembly connectable in sterile fluid communication with the formulation chamber of the formulation container or the intermediate formulation chamber of the intermediate formulation container, wherein the closed transfer filling assembly includes an electronic identifier, a valve or closure movable between a closed position sealing the interior of the closed transfer filling assembly and any formulation therein from the ambient atmosphere, and an open position allowing the flow of formulation therethrough.

49. An apparatus as defined in claim 48, wherein the closed transfer filling assembly is movable between a first position and a second position, in the first position the closed transfer filling assembly is locked in a closed position sealing any formulation therein from the ambient atmosphere, in the second position the closed transfer filling assembly is engageable with a dispensing container, the valve or closure of the closed transfer filling assembly is movable to the open position and connectable in sterile fluid communication with the interior of the dispensing container to allow the flow of sterile formulation through the closed transfer filling assembly and into the interior of the dispensing container.

50. An apparatus as defined in claim 49, wherein the closed transfer filling assembly comprises a piercing member, and each dispensing container includes an elastic septum, and in the second position of the closed transfer filling assembly, the piercing member is engageable with the elastic septum of a dispensing container, during movement between the first and second positions the piercing member penetrates the elastic septum and decontaminates the piercing member by physical interaction with the elastic septum, and the formulation is sterile transferred through the piercing member and into the dispensing container.

51. An apparatus as defined in claim 50, wherein each piercing member includes an outflow aperture and a closure movable between a closed position covering the outflow aperture, and an open position exposing the outflow aperture, in the first position of the closed transfer filling assembly the closure is locked in the closed position until the outflow aperture penetrates the septum, and in the second position after the outflow aperture penetrates the septum, the closure is unlocked with respect to the piercing member and at least one of the closure or piercing member is movable relative to the other to expose the outflow aperture and allow the flow of formulation therethrough and into the interior of the dispensing container.

52. An apparatus as defined in any one of claims 48 to 51, further comprising a plurality of closed transfer filling assemblies, each closed transfer filling assembly including a closed transfer filling head, a sterile connector, a flexible conduit sealingly connected between the respective closed transfer filling head and sterile connector, and an electronic identifier, wherein the sterile connector of each closed transfer filling head assembly is connectable to a sterile connector in fluid communication with the formulation chamber or the intermediate formulation chamber for the closed sterile transfer of formulation therethrough.

53. An apparatus as defined in any one of claims 47-52, further comprising an intermediate formulation container support, wherein the support is oriented at an acute angle with respect to a horizontal plane and defines an upper end and a lower end, the outlet port of the intermediate formulation container is located at the lower end of the support to direct the formulation within the intermediate formulation chamber toward the lower end and outlet port.

54. An apparatus as defined in any one of claims 47 to 53, wherein the intermediate formulation container includes a plurality of outlet ports in fluid communication with the intermediate formulation chamber, and a plurality of sterile connectors, wherein each sterile connector is connected in fluid communication with a respective outlet port and includes a respective electronic identifier.
